(19) United States
(12) Patent Application Publication
(10) Pub. No.: US 2015/0272935 A1
(43) Pub. Date: Oct. 1, 2015

(54) Use of a Factor Xa Inhibitor for Treating and Preventing Bleeding Events And Related Disorders in Patients Having Sensitivity to Vitamin K Antagonists Used As Anticoagulants

(71) Applicant: DAIICHI SANKYO COMPANY, LIMITED, Tokyo (JP)

(72) Inventors: Joseph Ray Walker, JR., Princeton Jct, NJ (US); Alexander Gene Vandell, South Plainfield, NJ (US)

(21) Appl. No.: 14/669,266

(22) Filed: Mar. 26, 2015

Related U.S. Application Data
(60) Provisional application No. 61/972,603, filed on Mar. 31, 2014.

(51) Int. Cl. A61K 31/437; C12Q 1/68; A61K 45/06

(52) U.S. Cl. CPC A61K 31/437; A61K 45/06; C12Q 1/6883; C12Q 2600/16; C12Q 2600/156; C12Q 2600/112

(57) ABSTRACT

The invention provides methods of treating or preventing bleeding events or over-anticoagulation in a subject in need thereof who is identified as having sensitivity to a vitamin K antagonist such as warfarin by administering to the subject a therapeutically effective amount of an FXa inhibitor, which can be a direct or indirect FXa inhibitor, or a warfarin or VKA alternative drug or compound. The direct FXa inhibitor can be the small molecule edoxaban p-toluenesulfonate monohydrate, edoxaban, or a pharmaceutically acceptable salt and/or hydrate thereof. In aspects, the subject is identified as having one or more genetic polymorphisms in genes CYP2C9 and/or VKORC1 resulting in loss of function, reduction in function, or aberrant function of these genes and/or their protein products, and sensitivity to warfarin. The invention provides methods of administering an FXa inhibitor or warfarin alternative to safely and effectively reduce, prevent, reduce the risk of, prevent the recurrence of, or prevent the risk of recurrence of, conditions such as embolism, thrombosis, thromboembolism, etc. in a subject who is in need of anticoagulant therapy and who is identified as having one or more genetic polymorphisms resulting in warfarin sensitivity.

### FIELD OF THE INVENTION

The invention relates generally to preventing or reducing bleeding risks in individuals identified as being sensitive to treatment with warfarin, as well as other vitamin K antagonists, and requiring anticoagulant therapy, by treating such individuals with a Factor Xa (FXa) inhibitor, or with a warfarin or vitamin K antagonist alternative drug or compound. The inventive treatment methods are particularly directed to those individuals identified as having one or more genetic polymorphisms in genes resulting in warfarin sensitivity. The invention further relates to methods of using an FXa inhibitor or warfarin or VKA alternative to treat and prevent thrombosis and embolism and related disorders or conditions in subjects identified as being warfarin sensitive.

### BACKGROUND OF THE INVENTION

The prevention of blood clot formation, expansion, and/or migration in the blood and blood vessels of individuals having certain types of medical conditions typically requires the use of anticoagulants. Patients who are afflicted with conditions such as irregular heartbeat, venous thrombosis, pulmonary embolism, prosthetic (replacement or mechanical) heart valves, and those who have suffered a heart attack, are frequently prescribed anticoagulant (“blood thinner”) medications to decrease the clotting ability of the blood. Warfarin (coumadin), a vitamin K antagonist (VKA), is a commonly prescribed anticoagulant which, while effective in reducing blood clotting, also suffers from a number of constraints that can place patients at risk for related, and potentially serious or fatal, bleeding events.

For sixty years, warfarin has been the most commonly used oral VKA for anticoagulant therapy. In the U.S. alone, over four million individuals are taking warfarin, which is effective for the primary and secondary prevention of venous thromboembolism (VTE), for the prevention of systemic embolism in patients with atrial fibrillation or prosthetic heart valves, for mitigating systemic embolism after myocardial infarction, and for reducing the risk of recurrent myocardial infarction.

Complications from bleeding in individuals undergoing anticoagulant therapy with warfarin is a major concern due to the narrow therapeutic range and the high degree of inter-individual variation in dose requirements. Therefore, accurate and appropriate dosing is critical for safely managing patients on this drug. Because non-genetic influences such as body size and age are poor predictors of an individual's dose requirement, there has been considerable investigation into the genetic influences on dose requirements for warfarin. Use of anticoagulants, such as the VKA warfarin, is hampered by a number of constraints, including highly variable response and the resultant need for close monitoring of patients and frequent dose adjustments.

Warfarin is metabolized primarily through oxidation in the liver by the CYP2C9 enzyme and exerts its anticoagulant effect by inhibiting the protein vitamin K epoxide reductase complex, subunit 1 (VKORC1). Three single nucleotide polymorphisms (SNPs), two in the CYP2C9 gene and one in the VKORC1 gene, have been found to play key roles in determining the effect of warfarin therapy on coagulation. Despite the ability to identify individuals who possess genetic variants of the CYP2C9 and VKORC1 genes, it is still difficult to determine appropriate and sustained doses of warfarin for those individuals who are sensitive or highly sensitive to warfarin's effects due to their particular CYP2C9 and VKORC1 genotypes. Bleeding complications from warfarin therapy constitute a leading drug-related reason for emergency room visits and are a significant reason for drug-related morbidity and mortality in the United States. (Hafner, J. W. et al., 2002, Ann. Emerg. Med., 39(3):258-266; Budnitz, D. S. et al., 2005, Ann. Emerg. Med., 45(2):197-206 and 2006, JAMA, 296(15):1858-1866; Levine, M. N. et al., 2001, Chest, 119(1)(Suppl.), 108S-121S; Wysowski, D. K. et al., 2007, Arch. Intern. Med., 167:1414-1419).

Thus, there is a need for additional and safe pharmaceutically acceptable drugs, agents and compounds to treat individuals who require anticoagulant therapy, but who suffer from, or who are at risk of suffering from, serious complications if prescribed warfarin or other VKA drugs due to their sensitivity to warfarin's metabolic effects. Novel and effective agents and methods for treating those individuals who are in need of anticoagulant therapy and possess functional variants of the CYP2C9 and VKORC1 genes, would greatly aid susceptible patients and the medical community, in view of the significant need. The present invention provides the means to address and fulfill such a need.

### SUMMARY OF THE INVENTION

The present invention is directed to methods of providing anticoagulant therapy for a subject who is in need thereof and who is sensitive to treatment with the anticoagulant warfarin or other VKAs. According to the methods of the invention, a Factor Xa (FXa) inhibitor, e.g., a direct or indirect FXa inhibitor, is administered in a therapeutically effective amount to the subject as an alternative to warfarin or a VKA drug or compound. In an embodiment, the FXa inhibitor is a direct FXa inhibitor, such as rivaroxaban, apixaban, and particularly, edoxaban, or a pharmaceutically acceptable salt and/or hydrate thereof. More particularly according to the invention, the subject who is warfarin sensitive is identified as having one or more genetic polymorphisms in the CYP2C9 and VKORC1 genes, which result in warfarin sensitivity in the subject. Such genetic polymorphisms in the CYP2C9 and VKORC1 genes can generate reduced function or hypo-functional variants of the CYP2C9 and VKORC1 genes or gene products, which play essential roles in the metabolism of S-warfarin and in the function of warfarin as a VKA, respectively. As reflected herein, the term Factor Xa inhibitor is abbreviated as an “FXa” inhibitor.

According to the methods of the invention, an FXa inhibitor is administered as an alternative to, or instead of, warfarin or other VKA drug or compound to a warfarin sensitive subject who is in need of anticoagulant therapy to treat, prevent, or reduce the risk of bleeding events in the subject. The FXa inhibitor can be a direct FXa inhibitor or an indirect FXa inhibitor. The bleeding events may encompass major bleeding, clinically overt bleeding or clinically relevant non-major (CRNM) bleeding events, e.g., as described herein and in Example 1.

In embodiments of the methods of the invention, a direct or an indirect FXa inhibitor is administered for anticoagulant treatment of a warfarin sensitive subject in need thereof instead of warfarin or a similarly acting VKA drug other than warfarin. In embodiments of the methods, a warfarin or a VKA alternative drug or compound is administered for anticoagulant treatment of a warfarin sensitive subject in need thereof instead of warfarin or VKA drug or compound. Examples of such warfarin or VKA alternatives, which may also be considered as “indirect FXa inhibitors,” include, without limitation, heparins, heparinoids, low molecular weight (LMW) heparins, ultra-low molecular weight heparins, low molecular weight lignins (LMWLs), thrombin (Factor IIa) inhibitors, or other direct thrombin inhibitors. In some embodiments, a warfarin or VKA alternative drug or compound can be a direct FXa inhibitor, an indirect FXa inhibitor, or other non-warfarin or VKA anticoagulant drug or compound.

It will be understood that, in general, the methods of the invention embrace the use of an anticoagulant that is not warfarin or a VKA drug or compound, but that is instead a warfarin or VKA alternative drug or compound, to treat warfarin sensitive individuals who are in need of anticoagulant therapy and who are identified through genotypic and/or phenotypic analysis as having one or more genetic polymorphisms affecting the function of the CYP2C9 and/or VKORC1 genes and/or their CYP2C9 and/or VKORC1 products, e.g., reduction in function variants. Thus, in their various embodiments, the methods of the invention embrace the use of a direct FXa inhibitor, an indirect FXa inhibitor, or a warfarin or VKA alternative drug or compound as anticoagulants to treat warfarin sensitive subjects. Non limiting examples of indirect FXa inhibitors, or warfarin or VKA alternative drugs and compounds, include heparins, heparinoids, low molecular weight (LMW) heparins, ultra-low molecular weight heparins, low molecular weight lignins (LMWLs), or thrombin/Factor IIa inhibitors as mentioned herein. In an embodiment, a direct FXa inhibitor is most suitable for use as an anticoagulant in accordance with the methods of the invention and serves as a warfarin or VKA alternative. In certain embodiments, the direct FXa inhibitor is edoxaban.

In particular aspects of the invention, the subject is a human patient suffering from a condition, disease, or disorder in which anticoagulant treatment is warranted or required, for example, conditions such as embolism, thrombosis, thromboembolism, heart disease, and the like, and the subject has, or is at risk of having, a bleeding event or over-anticoagulation associated with warfarin therapy or with VKA drug therapy. In embodiments, the subject is being treated for reducing the risk of stroke and/or systemic embolism in nonvalvular atrial fibrillation, for deep vein thrombosis (DVT), for pulmonary embolism (PE), for preventing or reducing the risk of recurrence of DVT and of PE, e.g., following initial treatment for DVT and/or PE, for DVT following hip or knee replacement surgery, or for prophylaxis of DVT following hip or knee replacement surgery. In an embodiment of the invention, the subject is determined to be sensitive, e.g., medium or highly sensitive, to warfarin based on the identification of the subject's genotypic or phenotypic sensitivity to warfarin as described herein. In an embodiment, the subject is determined to carry one or more genotypic polymorphisms in one or both of the CYP2C9 and VKORC1 genes resulting in reduction of function variants and warfarin sensitivity as described herein.

The methods of the invention involve the treatment of human patients requiring anticoagulant therapy with an effective amount of an FXa inhibitor, in particular, when the patients are identified as being sensitive to warfarin by having one or more genetic polymorphisms, mutations, or variations in the CYP2C9 and VKORC1 alleles that result in warfarin sensitivity. The patients may also be identified as having a warfarin sensitive phenotype by non-genetic or phenotypic methods or analyses in which genetic polymorphisms are manifested in a phenotypic effect, e.g., through CYP2C9 and VKORC1 protein loss of function, reduction in function, or abnormal function. For example, a phenotypic manifestation of one or more polymorphisms in CYP2C9 may be reflected by decreased or defective metabolism of S-warfarin by the encoded CYP2C9 protein product.

As referred to herein, the terms genetic polymorphism, genetic mutation and genetic variation are used interchangeably. In particular, subjects having one or more genetic polymorphisms in alleles of the CYP2C9 and/or VKORC1 genes, such as SNPs resulting in a loss of function, reduction in function, or aberrant function of the CYP2C9 and/or VKORC1 genes or gene products are warfarin sensitive and are prone to and/or are at risk of bleeding events or over-anticoagulation when treated with warfarin or another VKA. More specifically, in some embodiments, the one or more genetic polymorphisms in alleles of the CYP2C9 and/or VKORC1 genes are selected from a single nucleotide polymorphism (SNP) in the *2 allele of CYP2C9 (rs1799853); a SNP in the *3 allele of CYP2C9 (rs1057910); and/or a −1639G>A (rs9923231) SNP genetic polymorphism in the VKORC1 gene resulting in warfarin sensitivity, as described herein.

In another of its aspects, the invention provides a method of treating or preventing embolism, thrombosis, or thromboembolism in a subject in need thereof and identified as having one or more genetic polymorphisms in genes CYP2C9 and/or VKORC1 that result in warfarin sensitivity, in which the method comprises administering to the subject a therapeutically effective amount of a pharmaceutical composition comprising an FXa inhibitor.

In another of its aspects, the invention provides a method of treating thrombus, embolism, or thromboembolism in a subject to reduce the risk of a bleeding event, in which the method comprises a) assaying a biological sample from the subject to identify if the subject has sensitivity to warfarin; b) identifying the subject as having sensitivity to warfarin; and c) administering a therapeutic amount of an FXa inhibitor to the subject identified as having sensitivity to warfarin in step b).

In another of its aspects, the invention provides a method of treating a human subject in need thereof with a therapeutically effective amount of an FXa inhibitor to prevent, deter, or reduce the risk of a bleeding event or over-anticoagulation, wherein the subject is identified or characterized as carrying one or more genetic variations in the CYP2C9 and VKORC1 genes, which result in warfarin sensitivity in the subject, and further wherein the subject has a condition or disorder necessitating the use of an anticoagulant. In an embodiment, the FXa inhibitor is administered in a pharmaceutically acceptable composition.

In another of its aspects, the invention provides method of treating or preventing drug-induced bleeding events or over-anticoagulation in a subject in need thereof, the subject identified as having one or more genetic polymorphisms in genes CYP2C9 and/or VKORC1 that result in warfarin sensitivity, in which the method comprises administering to the subject an effective amount of an FXa inhibitor which is edoxaban or a pharmaceutically acceptable salt and/or hydrate thereof. In embodiments of the methods, the bleeding events are induced by VKAs used as anticoagulant drugs, such as warfarin or, for example, dicumarol, 4-hydroxycoumarin, phenprocoumon, indan-1,3-dione, acenocoumarol, anisindione, or other coumarin derivatives.

In another of its aspects, the invention provides a method of guiding anticoagulant therapy by determining whether to administer either 1) warfarin or another VKA, or 2) a warfarin or VKA alternative, or a direct FXa inhibitor, to a subject in need of anticoagulation therapy, in which the method comprises a) assaying a biological sample from the subject to identify genetic polymorphisms in genes CYP2C9 and/or VKORC1 indicative of warfarin sensitivity; b) identifying the subject as carrying one or more genetic polymorphisms in the CYP2C9 and/or VKORC1 genes indicative of warfarin sensitivity; c) if the subject is identified as carrying one or more of the genetic polymorphisms of step b), then administering a therapeutically effective amount of a composition comprising an FXa inhibitor or a warfarin or VKA alternative; or d) if the subject is identified as not carrying any of the genetic polymorphisms of step b), then administering to the subject a therapeutically effective amount of a composition comprising warfarin, or another pharmaceutically acceptable VKA drug, or an FXa inhibitor, or a warfarin or VKA alternative.

In another of its aspects, the invention provides a method of treating a human subject in need thereof with a therapeutically effective amount of a warfarin or VKA alternative drug or compound to prevent, deter, or reduce the risk of a bleeding event or over-anticoagulation, wherein the subject is identified or characterized as carrying one or more genetic variations in the CYP2C9 and VKORC1 genes, which result in warfarin sensitivity in the subject, and further wherein the subject has a condition or disorder necessitating the use of an anticoagulant. In an embodiment, the therapeutically effective amount of the warfarin or VKA alternative drug or compound is administered in a pharmaceutically acceptable composition. Such a warfarin or VKA alternative drug or compound may be, without limitation, a drug or compound which has been approved for indications such as, e.g., venous thromboembolism, embolism, thrombus, or post-surgical indications, etc., for which warfarin might be prescribed. Examples of warfarin or VKA alternative drugs or compounds include direct FXa inhibitors and indirect FXa inhibitors, e.g., anti-thrombin agents, and the like, rivaroxaban, or apixaban, as well as other agents as described herein.

In an aspect of the methods of the invention, the warfarin sensitive subject is preferably administered an FXa inhibitor, or warfarin or VKA alternative drug, as an initial treatment instead of warfarin when the subject requires warfarin therapy. By way of example, the clinical study described herein (Example 1) has determined that the safety and effectiveness of an FXa inhibitor such as edoxaban as an alternative to warfarin for a warfarin sensitive subject who is prescribed anticoagulant therapy is evident during at least the first 90 days after the subject has taken warfarin therapy. Notwithstanding, initial and sustained treatment of warfarin sensitive subjects with an FXa inhibitor, or a warfarin or VKA alternative, is also highly beneficial in terms of safety and effectiveness for such subjects. Accordingly, treatment of a warfarin sensitive subject with an FXa inhibitor or warfarin or VKA alternative is recommended as an initial and/or early treatment for such a subject in need of anticoagulant therapy, rather than starting a warfarin sensitive subject on warfarin therapy and risking the occurrence of a mild, moderate, or severe bleeding event in the subject, particularly during at least the first 90 days of warfarin treatment. The methods of the invention offer the advantage that an FXa inhibitor or warfarin or VKA alternative drug provides greater safety and treatment benefit for a warfarin sensitive subject, particularly a subject determined to have medium or high sensitivity to warfarin, in preventing or reducing, or reducing the risk of, bleeding events or over-anticoagulation frequently associated with warfarin use in such subjects.

In an aspect of each of the above methods, the dosage of the FXa inhibitor edoxaban, or a pharmaceutically acceptable salt and/or hydrate thereof, administered is from 0.1 mg to at least 90 mg per day; or from 5 mg to 90 mg per day; or from 30 mg to 60 mg per day, or from 30 mg to 75 mg per day; or from 15 mg per day to 60 mg per day. In an aspect of each of the above methods, the therapeutically effective amount of edoxaban is 60 mg per day. In an aspect of each of the above methods, the therapeutically effective amount of edoxaban is 30 mg per day. In an aspect of each of the above methods, the therapeutically effective amount of the anticoagulant rivaroxaban is 10 mg per day, with or without food; 15 mg or 20 mg per day with food. The effective dose of rivaroxaban is specific to the indication being treated. In an aspect of each of the above methods, the therapeutically effective amount of the anticoagulant apixaban is 2.5 mg, twice daily. In another aspect of each of the above methods, the administering comprises oral administration. In another aspect of each of the above methods, the subject suffers from, or is afflicted with a condition or disorder for which anticoagulant therapy is prescribed and administered as described herein.

In an aspect of each of the above methods, the subject is a human subject or human patient. In an aspect of each of the above methods, the FXa inhibitor is a direct or indirect inhibitor of FXa. In an aspect of each of the above methods, the FXa inhibitor is a direct inhibitor of FXa. In an aspect of each of the above methods, the FXa inhibitor is the small molecule edoxaban, or a pharmaceutically acceptable salt and/or hydrate thereof. In an aspect of each of the above methods, the FXa inhibitor is edoxaban p-toluenesulfonate monohydrate and is also referred to as edoxaban tosylate monohydrate or edoxaban. In an aspect of each of the above methods of the invention, the direct FXa inhibitor is administered in solid form, such as a tablet, pill, capsule, and the like. In an aspect of each of the above methods, the direct FXa inhibitor is orally bioavailable and is orally administered to a subject in need thereof. In a specific aspect of each of the above methods of the invention, the FXa inhibitor is in a solid dosage form. It will be understood by those having skill in the art, even when not explicitly stated herein, that a direct FXa inhibitor may be used in a form which is a pharmaceutically acceptable salt and/or hydrate of the FXa inhibitor molecule or compound.

In aspects of each of the above methods, a subject identified as having one or more genetic polymorphisms in CYP2C9 and/or VKORC1 genes resulting in warfarin sensitivity can be medium sensitive or highly sensitive to bleeding or over-anticoagulation associated with warfarin treatment. In particular aspects of the above methods, the subject has one or more genetic polymorphisms in alleles of the CYP2C9 gene selected from a single nucleotide polymorphism (SNP) in the *2 allele of CYP2C9 (rs1799853), a SNP in the *3 allele of CYP2C9 (rs1057910); and/or a −1639G>A (rs9923231) SNP genetic polymorphism in the VKORC1 gene, the SNPs being associated with warfarin sensitivity in the subject.

In particular aspects of each of the above methods, the subject has medium sensitivity to warfarin; such medium warfarin sensitivity can be identified by the subject's having CYP2C9 and VKORC1 allelic genotypes (also termed “haplotypes”) selected from a *1/*1 genotype in CYP2C9 and an A/A genotype in VKORC1; a *1/*2 genotype in CYP2C9 and an A/G genotype in VKORC1; a *1/*2 genotype in CYP2C9 and an A/A genotype in VKORC1; a *1/*3 genotype in CYP2C9 and a G/G genotype in VKORC1; a *1/*3 genotype in CYP2C9 and an A/G genotype in VKORC1; a *2/*2 genotype in CYP2C9 and a G/G genotype in VKORC1; a *2/*2 genotype in CYP2C9 and an A/G genotype in VKORC1; or a *2/*3 genotype in CYP2C9 and a G/G genotype in VKORC1.

In other particular aspects of each of the above methods, the subject has high sensitivity to warfarin; such high warfarin sensitivity can be identified by the subject's having CYP2C9 and VKORC1 allelic genotypes (also termed “haplotypes”) selected from a *1/*3 genotype in CYP2C9 and an A/A genotype in VKORC1; a *2/*2 genotype in CYP2C9 and an A/A genotype in VKORC1; a *2/*3 genotype in CYP2C9 and an A/G genotype in VKORC1; a *2/*3 genotype in CYP2C9 and an A/A genotype in VKORC1; a *3/*3 genotype in CYP2C9 and a G/G genotype in VKORC1; a *3/*3 genotype in CYP2C9 and an A/G genotype in VKORC1; or a *3/*3 genotype in CYP2C9 and an A/A genotype in VKORC1. In a particular aspect of the above methods, a subject having medium or high warfarin sensitivity is identified as having a non-polymorphic (normal or wild-type) CYPC29 genotype and an A/A VKORC1 genotype.

In embodiments of each of the above methods, the FXa inhibitor is a direct Factor Xa inhibitor selected from edoxaban, rivaroxaban, LY517717, apixaban, 813893, betrixaban, AVE-3247, EMD-503982, WX-FX4, or a pharmaceutically acceptable salt and/or hydrate thereof. In other embodiments of each of the above methods, the FXa inhibitor is an indirect Factor Xa inhibitor, or a warfarin or VKA alternative drug or compound, selected from heparins, heparinoids, low molecular weight (LMW) heparins, ultra-low molecular weight heparins, low molecular weight lignins (LMWLs), or direct thrombin/Factor IIa inhibitors, such as dabigatran, as described herein.

In further aspects of each of the above methods, the FXa inhibitor is administered in combination with another therapeutic, drug, or bioactive agent. In some embodiments, the FXa inhibitor is administered or used concomitantly with other drugs such as statins, e.g., atorvastatin; P-gp substrates, e.g., digoxin; antiplatelets; antithrombotic agents; fibrinolytics; non-steroidal anti-inflammatory drugs, e.g., acetylsalicylic acid (aspirin) or naproxen; proton pump inhibitors (PPIs), e.g., esomeprazole, or other agents as described herein. In embodiments, the therapeutic or bioactive agent is not warfarin or other VKA drug or compound.

In other aspects of each of the above methods, the warfarin sensitive subject to be treated with an FXa inhibitor according to the invention has, or is at risk of having, a condition or disorder selected from one or more of venous thromboembolism (VTE), deep vein thrombosis pulmonary embolism, embolism, thromboembolism (TE), and venous thrombosis (VT), cerebral infarction, cerebral embolism, myocardial infarction, angina pectoris, pulmonary infarction, pulmonary embolism (PE), Buerger's disease, deep venous thrombosis (DVT), disseminated intravascular coagulation syndrome, thrombus formation after surgery, thrombus formation after valve or joint replacement, thrombus formation and reocclusion after angioplasty, systemic inflammatory response syndrome (SIRS), multiple organ dysfunction syndrome (MODS), thrombus formation during extracorporeal circulation, or blood clotting. In an embodiment, warfarin or another VKA is not administered to the subject. In other aspects of each of the above methods, the subject is being treated for reducing the risk of stroke and systemic embolism associated with nonvalvular atrial fibrillation, for deep vein thrombosis (DVT), for pulmonary embolism (PE), for preventing or reducing the risk of recurrence of DVT and PE, e.g., following initial treatment for DVT and/or PE, for DVT following hip or knee replacement surgery, or for prophylaxis of DVT following hip or knee replacement surgery.

In embodiments of the methods of the invention, a subject in need thereof is determined or identified to be warfarin sensitive via genotypic or phenotypic testing or analysis. For example, and without limitation, warfarin sensitivity can be determined in a subject through genetic or nucleic acid-based testing or analysis, including genotypic analysis, whole genome sequencing, transcriptome sequencing, in which genetic variation or polymorphisms are identified or determined. In addition, warfarin sensitivity can be determined in a subject through phenotypic analysis or assays in which loss of function, reduction in function, or aberrant function of the gene products of one or both of the CYP2C9 and VKORC1 genes is identified or assessed relative to a normal or wild-type control. In such cases, a warfarin sensitive phenotype is determined not by genetic analysis. but by non-genetic methods as practiced in the art, such as in vitro or in vivo phenotypic screens. For example, cell based assays can be performed in which loss of function, reduction in function, or aberrant function of the CYP2C9 and/or VKORC1 proteins is quantitatively evaluated or assessed. Such assays can involve testing for CYP2C9 and/or VKORC1 phenotypes that are warfarin sensitive; or determining that a CYP2C9 protein metabolizes warfarin at a decreased rate; and/or determining that a VKORC1 product has an increased or decreased enzyme activity. In addition, a combination of genotypic and phenotypic determinations can be carried out to identify individuals as warfarin sensitive, as well as their levels of response or sensitivity to warfarin, e.g., medium or high sensitivity to warfarin.

The foregoing and other aspects, features and advantages of the invention and its embodiments will become apparent in the descriptions of the accompanying drawings and detailed description provided herein.

### BRIEF DESCRIPTION OF THE DRAWINGS

FIG. 1 shows a plot of overt bleeding events during the first 90 days of treatment with anticoagulant among warfarin-treated patients across genotypes reflecting warfarin sensitivity response in subjects (3-bin). The cumulative incidence of any overt bleeding (ISTH major, clinically relevant non-major (CRNM) and minor bleeding) is shown in patients identified as normal responders, sensitive responders and highly sensitive responders based on genotype. The results indicate that sensitive responders experienced a 30% higher risk of bleeding compared to normal responders, while highly sensitive responders demonstrated a greater than 2.5-fold increased risk of bleeding.

FIGS. 2A and 2B show plots of safety outcomes (bleeding events) during the first 90 days (FIG. 2A) and beyond 90 days (FIG. 2B) among warfarin and edoxaban treated patients across genotypes reflecting warfarin sensitivity response in subjects (3-bin). Event rates, hazard ratio (HR), (95% confidence interval, CI), and interaction terms are presented across genotypes. The circles indicate the HR and the horizontal lines represent the 95% confidence boundaries. During the first 90 days, when compared to warfarin, the FXa inhibitor edoxaban as treatment drug resulted in a particularly decreased risk of bleeding in sensitive and highly sensitive responders in this early treatment period. (FIG. 2A). The pharmacogenetic analysis demonstrates that the relative safety of the FXa inhibitor edoxaban versus warfarin was particularly evident during the early time period among sensitive and highly sensitive responders. Beyond 90 days, the beneficial safety profile of the FXa inhibitor edoxaban versus warfarin was evident across all the genetic categories. (FIG. 2B).

### DETAILED DESCRIPTION OF THE INVENTION

The invention generally provides methods of treating, preventing, or reducing the risk or incidence of, bleeding or over-anticoagulation events in individuals who are identified or characterized as being sensitive to warfarin therapy and are in need of anticoagulation therapy. The identification of such warfarin sensitive individuals is not intended to be limiting and can be determined, for example, by genotypic or phenotypic testing or analysis, involving genetic polymorphism (SNP) analysis, whole genome or transcriptome sequencing analysis, or loss of function analysis or reduction in function analysis via in vitro or in vivo phenotypic screening assays.

In an embodiment of the invention, individuals who are sensitive to warfarin are identified through genotypic analysis or screening as possessing certain genetic polymorphisms in the CYP2C9 and VKORC1 genes related to vitamin K metabolism, where the individuals can be classified as sensitive, e.g., medium sensitive or highly sensitive, responders to the anticoagulant warfarin. In general, genetic variability in the CYP2C9 and VKORC1 genes translates into variability in a subject's pharmacological response to warfarin. More specifically, such genetic variability commonly results from single nucleotide polymorphisms (SNPs) in the CYP2C9 and/or VKORC1 genes, which can be determined by conventional genotyping analysis of patients who require anticoagulant therapy. Individuals having one or more genetic polymorphisms in alleles of the CYP2C9 and/or VKORC1 genes, such as SNPs resulting in a loss of function, reduction in function, or aberrant function of the CYP2C9 and/or VKORC1 genes and/or their gene products are identified as warfarin sensitive and are prone to and/or at risk of bleeding events or over-anticoagulation when treated with warfarin or another VKA.

Examples of typical, noninvasive genetic testing methods for genotype identification, determination, or detection include, without limitation, assaying peripheral blood, a buccal swab, or hair follicle sample as a source of DNA for genetic analysis, or of cellular material for performing in vitro or in vivo phenotypic assays. Phenotypic assays can detect or determine a loss of, reduction in, or aberrant function or activity in the CYP2C9 and/or VKORC1 gene products. Specifically, for example, the *2 and *3 variants in the CYP2C9 gene result in reduced catalytic activity of the CYP2C9 product, and hence, reduced metabolism of the more active S-enantiomer of warfarin (Lee C. R. et al., 2002, Pharmacogenetics, 12:251-63); and the −1639G>A variant in VKORC1 alters a transcription factor binding site and hence leads to reduced levels of the molecular target of warfarin. (Rieder, M. J., 2005, N Engl J Med, 352:2285-93).

Warfarin (also known by the brand names Coumadin, Jantoven, Marevan, Uniwarfin, Warf) is an anticoagulant conventionally used in the prevention of thrombosis and thromboembolism, i.e., the formation of blood clots in the blood vessels and their migration elsewhere in the body, respectively, in patients having disorders, conditions and diseases in which blood clots and their migration can occur. Despite its effectiveness and widespread use, treatment with warfarin has numerous disadvantages. Many commonly used medications interact with warfarin, as do some foods (particularly leafy vegetable foods or “greens,” since these typically contain large amounts of vitamin K1). In addition, the activity of warfarin has to be monitored by blood testing for the international normalized ratio (INR) to ensure that patients are receiving an adequate yet safe dose. A high INR predisposes a patient to a high risk of bleeding, while an INR below the therapeutic target indicates that the dose of warfarin is insufficient to protect against thromboembolic events. In addition, warfarin can cause severe bleeding that can be life-threatening and even cause death. Frequently, appropriate and thorough monitoring of patients for INR is suboptimal, thus leading to patients having bleeding events or being over-anticoagulated or at risk thereof. A bleeding event, or adverse bleeding event, is often more likely during warfarin treatment for people over 65 years of age, and it is also more likely during about the first 30 to 90 days of warfarin treatment. Bleeding is also more likely to occur in people who take high doses of warfarin and/or for a long period of time.

Because of warfarin's wide inter-individual variation in dosage requirements, as well as its narrow therapeutic index, accurate and appropriate dosing is critical for safely managing patients who take this drug. Individual genetic influences on warfarin's dose requirements have become important considerations for dosing patients who require its use, especially in view of the overall failure of typical, non-genetic factors, such as body mass, age, weight, etc., to predict suitable warfarin dosage requirements.

In embodiments of the invention, methods are provided in which subjects who are genotypically identified as being sensitive to the well-known VKA warfarin and who are in need of anticoagulation treatment or therapy are administered a therapeutically effective amount of an FXa inhibitor, e.g., edoxaban or a pharmaceutically acceptable salt and/or hydrate thereof, instead of warfarin or a non-warfarin VKA, for anticoagulant treatment or therapy. Nonlimiting examples of other VKAs include dicoumarol, phenindione, phenprocoumon, acenocoumarol, ethyl biscoumacetate, clorindione, diphenadione, tioclomarol and fluindione. Such VKAs used as anticoagulation drugs may also result in bleeding events in subjects who are identified as being genotypically sensitive to the VKA warfarin, as described herein. Embodiments of the present methods embrace treating subjects identified as being VKA sensitive with a Factor Xa inhibitor, such as a direct Factor Xa inhibitor, e.g., edoxaban, as an alternative to treating with warfarin or other VKA drug.

### Genotypes and Genotypic Variability Related to Warfarin Sensitivity

As will be appreciated by the skilled practitioner, genetic variation (in individuals and in populations) is brought about by mutation, which is a permanent change in the chemical structure of a DNA gene sequence. Gene mutations range in size from a single DNA nucleotide base to a large segment of a chromosome; mutations may be inherited or germ line in origin, or they may be somatic in origin, acquired during an individual's lifetime. While some genetic changes are very rare, others are common in the population. Genetic changes that occur in more than 1 percent of the population are termed polymorphisms and are common enough to be considered a normal variation in the DNA. Polymorphisms are responsible for many of the normal differences among people, such as eye and hair color and blood type. Although many polymorphisms have no negative effects on a person's health, some of these genetic variations may influence the risk of developing certain disorders or may correlate with a disease, drug response, and other phenotypes. Generally, a polymorphism involves one of two or more variants of a particular DNA sequence, with the most common type of polymorphism involving variation at a single base pair, such as a SNP. Herein, the terms genetic polymorphism, genetic mutation and genetic variation are used interchangeably. A functional genetic variant reflects a gene product encoded by a gene having a genetic polymorphism, mutation, or variation, where the phenotype, or function of the variant product, may be different, or may result in a different effect or response, from that of a normal, non-variant or non-mutant gene product. Frequently, a variant product exhibits abnormal, aberrant, subpar, or no activity or function relative to the normal product.

The CYP2C9 gene product, i.e., cytochrome P450 isozyme 2C9, is the primary enzyme involved in the metabolism and subsequent inactivation of S-warfarin in the liver. Vitamin K epoxide reductase (VKOR), the enzyme that catalyzes the reduction of vitamin K epoxide into a reduced form of vitamin K, is the target of warfarin's activity. Warfarin interferes with the vitamin K cycle by inhibiting the vitamin K epoxide reductase complex, subunit 1 (VKORC1) that generates a reduced form of vitamin K from the epoxide form of vitamin K. Vitamin K, in turn, is involved in the biosynthesis and function of coagulation factors, e.g., descarboxy-prothrombin and prothrombin, made in the liver. Warfarin's anticoagulation effects are linked to its action as an inhibitor of the VKORC1 reductase enzyme subunit.

In general, one or more genetic polymorphisms, e.g., SNPs, in either the CYP2C9 or the VKORC1 gene, or in both of these genes, are termed “reduction in function” SNPs and affect the CYP2C9 and/or VKORC1 gene products in subjects having such polymorphisms. The identification or determination of such reduction in function SNPs result in warfarin sensitivity in a subject and can place the subject at risk of bleeding events or over-anticoagulation if given warfarin, or other VKA drugs. In certain embodiments of the invention, three SNPs, two in the CYP2C9 gene and one in the VKORC1 gene, have been found to play key roles in determining the effect of warfarin therapy on coagulation. The SNPs in CYP2C9 include rs1057910 and rs1799853; a VKORC1 SNP is rs8050894. Because a tight linkage exists between several VKORC1 SNPs, the rs9923231 SNP (also known as −1639G>A) can be equivalent to rs8050894 for testing purposes. Since 2007, the FDA has recommended genetic testing or screening for SNPs in CYP2C9 and VKORC1 genes in an effort for medical practitioners to arrive at and manage warfarin dose determinations in individuals having conditions that are treated with anticoagulants such as warfarin.

As appreciated by practitioners in the art, the nomenclature for the CYP2C9 SNPs resulting in warfarin sensitivity and identifiable following genetic analysis is as follows: the normal or wild-type CYP2C9 allele is referred to as *1 (“star 1”), the two polymorphic allelic versions are *2 (“star 2”) and *3 (“star 3”), and each individual can carry any two versions of the SNP. For example, an individual with two normal copies of the CYP2C9 gene is designated as *1/*1; an individual with only one polymorphism could be *1/*2 or *1/*3, and an individual with both polymorphisms could be *2/*3, *2/*2, or *3/*3, which are also termed SNP genotypes or haplotypes of the CYP2C9 gene. In general, the prevalence of each variant genotype varies by race; 10% and 6% of Caucasians carry the *2 and *3 variants, respectively, but both variants are rare (e.g., <2%) in individuals of African or Asian descent. (Au N., and Rettie A. E., 2008, Drug Metab Rev., 40(2):355-75). In general, CYP2C9*1 metabolizes warfarin normally; CYP2C9*2 reduces warfarin metabolism by about 30%; and CYP2C9*3 reduces warfarin metabolism by about 90%. Because patients who have been genotyped as carrying the *2 or *3 variants of CYP2C9 metabolize warfarin less efficiently, the drug will remain in circulation longer after administration; therefore, lower warfarin doses are needed to achieve optimal anticoagulation.

In the VKORC1 −1639 SNP, which occurs in the promoter region of the VKORC1 gene, the common G allele is replaced by the A allele. The potential genotypes (also called “haplotypes”) for VKORC1 include G/G, G/A, or A/A, with the G/G and A/A genotype populations representing about 85% of individuals and the A/A genotype population representing about 15% of individuals. Because individuals with an A allele (or the “A genotype”) produce less VKORC1 product than do those with the G allele (or the “non-A genotype”), lower warfarin doses are needed to inhibit VKORC1 and to produce an anticoagulant effect in carriers of the A allele. The prevalence of the variants of VKORC1 also varies by ethnicity, with approximately 37% of Caucasians (European-Americans) and approximately 14% of African Americans carrying the A allele. (Rieder, M. J. et al., 2005, N Engl J Med., 352(22):2285-93).

Together, the three SNPs in alleles of the CYP2C9 and VKORC1 genes play key roles in determining the dose of warfarin required to produce a therapeutic International Normalization Ratio (INR), (typically 2.0 to 3.0); the risk of bleeding or of producing an INR indicative of over-anticoagulation (>4.0); and the time required to achieve a stable therapeutic dose. A combination of the two CYP2C9 variants (*2 and *3) with the VKORC1 −1639G>A promoter mutation accounts for ˜40-63% of the variability in therapeutic warfarin dose. For example, individuals who carry the CYP2C9*2 and CYP2C9*3 polymorphisms require, on average, about a 19% and 33% reduction, respectively, per allele in warfarin dose versus those who carry the *1 allele. Individuals who carry the VKORC1 A allele typically require, on average, a 28% reduction per allele in their warfarin dose compared with individuals who do not carry the allele. According to the present invention, the VKORC1−1639G>A SNP can adequately define a warfarin sensitive phenotype of an individual. Fₒᵣ example, individuals carrying two VKORC1 A alleles, “AA genotype” individuals, have lower hepatic VKORC1 mRNA expression and require much lower doses of warfarin to achieve a therapeutic INR.

Based on the foregoing, the use of standard or conventional warfarin dosing algorithms for patients harboring these genetic variants can lead to adverse, sometimes seriously adverse, clinical and laboratory outcomes because of a patient's genetically mediated sensitivity to the drug. For example, on average, standard dosing algorithms can lead to a 2- to 3-fold increased risk of serious or life threatening bleeding, or an out-of-range INR (≧ or >4.0), in carriers of the *2 or *3 alleles of CYP2C9. (Higashi, M. K., et al., 2002, JAMA, 287(13):1690-1698). Similarly, carriers of the VKORC1 A allele are also at a 2- to 3-fold higher risk of an INR >4.0 during initiation of warfarin therapy when standard dosing algorithms are used. (Schwarz, U. I. et al., 2008, N Engl J Med., 358(10):999-1008). As a result of the sensitivity of these patients to warfarin and the additional dose adjustments required, the time required to achieve a “stable” INR between 2.0 and 3.0 is significantly delayed in carriers of all three SNPs. Thus, the use of a combination of genetic and clinical factors to predict the maintenance warfarin dose appears to be more accurate than using clinical factors alone. The incorporation of the various factors that influence warfarin dose can be difficult to implement clinically; therefore, online warfarin dosing calculators, such as the one found, for example, at http://www.WarfarinDosing.org and supported by Barnes-Jewish Hospital at Washington University Medical Center, are available to aid in determining the appropriate dose adjustments. (Gage, B. F. et al., 2008, Clin Pharmacol Ther., 84(3):326-331).

While the above-described genetic polymorphisms, namely, two SNPs in CYP2C9 and one SNP in VKORC1, can be readily identified as reduction in function SNPs resulting in warfarin sensitivity and bleeding risk in individuals harboring such polymorphisms, other reduction in function polymorphisms (SNPs) in the CYP2C9 and/or VKORC1 genes can also serve to identify warfarin sensitive individuals who are at risk of bleeding events if treated with warfarin, or other VKA drugs, and are thus embraced by the methods of the present invention. Accordingly, the identification of a number of polymorphisms (SNPs) in CYP2C9 may result in protein changes and have functional impact on the warfarin sensitivity of individuals who carry these polymorphisms. A number of SNPs in CYP2C9 alleles, e.g., CYP2C9*1 to CYP2C9*58, have been identified through genetic testing and sequence analysis and can be used to characterize an individual's sensitivity to warfarin or other VKA drug treatment. Many of the protein products encoded by the polymorphic alleles exhibit altered functional activity relative to products encoded by normal alleles. CYP2C9 alleles, including genotypic and phenotypic information related to the alleles and gene products can be found in the pharmacogenetic literature, for example, at http://www.Cypalleles.ki.se/cyp2c9.htm and in a review of Cytochrome P450 2C9 polymorphisms by Lee, C. R. et al. (2002, Pharmacogenetics, 12(3):251-263), the contents of which are incorporated by reference herein. Thus, an individual's sensitivity to warfarin can be ascertained by determining the presence of one or a combination of a number of different SNPs in CYP2C9 alleles and/or their protein products, that have been identified and associated with response to warfarin.

Similarly, based on genomic sequence analyses of patient clinical samples, several VKORC1 genotypic SNP variants have been identified and reported, in addition to the −1639 SNP described herein, which can also confer warfarin sensitivity on individuals carrying such variants or polymorphisms. More specifically, the 11 kb genomic region of VKORC1 was sequenced, including 5 kb in the upstream promoter region, 4.2 kb of the intron/exon sequence, and 2 kb of the downstream region. Ten SNPs (at positions 381, 861, 2653, 3673, 5808, 6009, 6484, 6853, 7566 and 9041 of the VKORC1 reference sequence, GenBank Accession No. AY587020) were identified as being associated with response to warfarin dose. See, Rieder, M. J. et al., 2005, N. Eng J. Med., 352(22):2285-2293. Accordingly, the methods of the invention contemplate treating subjects in need of anticoagulant therapy and identified as having one or more polymorphisms in VKORC1 that correlate with and/or result in a warfarin sensitive phenotype, including the −1639 SNP as well as other variants and combinations thereof, with an FXa inhibitor, or a warfarin or VKA alternative drug for safe and effective anticoagulant treatment.

The associated and potentially serious bleeding risks resulting from giving increased and/or suboptimal warfarin doses to patients who suffer from conditions, diseases and dysfunctions requiring the use of anticoagulants, e.g., to treat or prevent thrombosis and embolism when such patients are identified as being warfarin sensitive and/or having the described genetic polymorphisms in the genes CYP2C9 and VKORC1, necessitates the provision of new and alternative methods and pharmaceutically acceptable agents that achieve better, safer, and optimally improved results in treating, preventing, or reducing bleeding events, blood clot formation and/or migration in such patients, without the risks and limitations associated with the use of warfarin and other, similarly acting VKA drugs.

### Factor Xa (FXa) Inhibitors

In an embodiment of the described methods, the FXa inhibitor is a small molecule inhibitor of the FXa serine protease. In an embodiment, the FXa inhibitor is a direct inhibitor of FXa activity. In an embodiment, the FXa inhibitor is an indirect inhibitor of FXa activity, e.g., by interacting with prothrombin. In an embodiment, the FXa inhibitor is a direct inhibitor of FXa activity that exhibits both FXa inhibiting effects and has antithrombotic, as well as anticoagulant effects.

FXa, a key serine protease coagulation factor, plays a vital role in thrombosis and hemostasis by enzymatically cleaving its substrate, prothrombin, to produce thrombin in the coagulation cascade, thereby regulating the generation of this important procoagulant enzyme. In addition, FXa contributes to additional physiological and pathophysiological mechanisms by eliciting a number of critical cellular responses, such as cytokine release, adhesion molecule expression, tissue factor (TF) expression, and cell proliferation, which are mediated by specific receptors and are involved with intracellular and/or extracellular signaling molecules and mediators. The binding of FXa to cells and the resulting stimulation or activation of cellular events positions FXa as a contributing force in a variety of diseases and pathologies. Regardless of the disease, the inhibition of FXa in the coagulation cascade prolongs clotting time and potentially reduces the risk of spontaneous or induced thrombus formation, thereby having a beneficial effect on patient treatment.

In embodiments, the FXa inhibitor for use according to the methods of the invention is selected from direct (e.g., direct binding to FXa) or indirect (e.g. activity dependent upon antithrombin) inhibitors of FXa. In an embodiment, the FXa inhibitor is a direct FXa inhibitor which is orally active. Nonlimiting examples of direct FXa inhibitors suitable for use in the methods include edoxaban (Daiichi Sankyo Co., Ltd.), rivaroxaban (Bayer Healthcare AG and Janssen Pharmaceuticals, Inc.), LY517717 (Lilly), apixaban (Bristol-Myers Squibb Company), 813893 (GlaxoSmithKline), betrixaban, AVE-3247, EMD-503982, the 3-amidoinophenylalanine-type FXa inhibitor, WX-FX4, or a pharmaceutically acceptable salt and/or hydrate thereof. Non-limiting examples of indirect FXa inhibitors suitable for use in the methods include heparins, heparinoids, low molecular weight (LMW) heparins (e.g., dalteparin, tinzaparin, reviparin, nadroparin, ardeparin, certoparin, parnaparin, or M118 (Momenta Therapeutics)), ultra-low molecular weight heparins (e.g., Semuloparin sodium (AVE5026; Sanofi-Aventis)), low molecular weight lignins (LMWLs), fondaparinux (ARIXTRA®) (GlaxoSmithKline), Idraparinux sodium (Sanofi-Aventis and Organon), or thrombin/Factor IIa inhibitors (e.g., bivalirudin, argatroban, desirudin, lepirudin, xemelagatran, or dabigatran etexilate mesylate (PRADAXA®, Boehringer Ingelheim, Ridgefield, Conn.)).

In a particular embodiment, the FXa inhibitor is the direct FXa inhibitor edoxaban p-toluenesulfonate monohydrate or “edoxaban” herein. Edoxaban p-toluenesulfonate monohydrate is a potent, orally active, selective, direct and reversible inhibitor of FXa, manufactured by Daiichi Sankyo Co., Ltd., Japan. (See, e.g., T. Furugohri et al., 2008, “DU-176b, a potent and orally active FXa inhibitor: in vitro and in vivo pharmacological profiles”, J. Thrombosis and Haemostasis, 6:1542-49; and U.S. Pat. No. 7,365,205, the contents of which are incorporated by reference herein in their entirety).

In a particular embodiment, the direct FXa inhibitor is N¹-(5-chloropyridin-2-yl)-N²-4-[(dimethylamino)carbonyl]-2-{[(5-methyl-4,5,6,7-tetrahydrothiazolo[5,4-c]pyridin-2-yl)carbonyl]amino}cyclohexyl)ethanediamide having the structure:

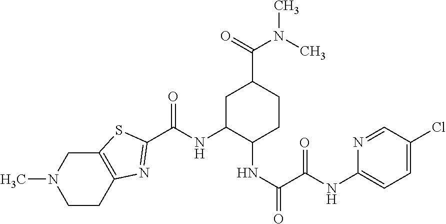 [SMILES: CN1CCc2nc(C(=O)NC3CC(C(=O)N(C)C)CCC3NC(=O)C(=O)Nc3ccc(Cl)cn3)sc2C1]

The FXa inhibitor may be a pharmaceutically acceptable salt and/or hydrate of N¹-(5-chloropyridin-2-yl)-N²-4-[(dimethylamino)carbonyl]-2-{[(5-methyl-4,5,6,7-tetrahydrothiazolo[5,4-c]pyridin-2-yl)carbonyl]amino}cyclohexyl)ethanediamide, such as a p-toluenesulfonate salt, and/or a hydrate thereof, particularly a monohydrate.

Other pharmaceutically acceptable salts include conventional, relatively non-toxic, inorganic or organic addition salts or quaternary ammonium salts of the parent compound formed, for example, from non-toxic inorganic or organic acids. For example, such conventional non-toxic salts include those derived from inorganic acids such as hydrochloric acid, hydrobromic acid, sulfuric acid, sulfamic acid, phosphoric acid, nitric acid and the like; and those prepared from organic acids such as acetic acid, propionic acid, succinic acid, glycolic acid, stearic acid, lactic acid, maleic acid, tartaric acid, citric acid, ascorbic acid, pamoic acid, maleic acid, hydroxymaleic acid, phenylacetic acid, glutamic acid, benzoic acid, valeric acid, oleic acid, salicylic acid, sulfanilic acid, 2-acetoxybenzoic acid, fumaric acid, p-toluenesulfonic acid, methanesulfonic acid, ethane disulfonic acid, oxalic acid, ethylenediaminetetraacetic acid, formic acid, benzene sulfonic acid, naphthalene-2-sulfonic acid, 3-hydroxy-2-naphthalenecarboxylic acid, and the like. (See, also, e.g., S. M. Barge et al. 1977, Pharmaceutical Salts, J. Pharm. Sci., 66:1-19). Such physiologically acceptable salts are prepared by methods known in the art, e.g., by dissolving the free amine bases with an excess of the acid in aqueous alcohol, or by neutralizing a free carboxylic acid with an alkali metal base, such as a hydroxide, or with an amine.

In an embodiment, the methods of the invention embrace the use of stereoisomers of N¹-(5-chloropyridin-2-yl)-N²-4-[(dimethylamino)carbonyl]-2-{[(5-methyl-4,5,6,7-tetrahydrothiazolo[5,4-c]pyridin-2-yl)carbonyl]amino}cyclohexyl)ethanediamide, in particular N¹-(5-chloropyridin-2-yl)-N² (1S,2R,4 S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide, which has the structure reproduced below.

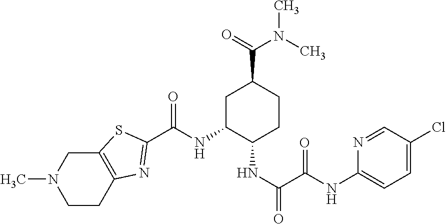 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]

The FXa inhibitor may also include pharmaceutically acceptable salts and/or hydrates of N¹-(5-chloropyridin-2-yl)-N²(1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide, particularly, p-toluenesulfonate salts and either the anhydrous or the monohydrate forms.

In a specific embodiment, the FXa inhibitor is edoxaban p-toluenesulfonate monohydrate, which has the formula: N¹-(5-chloropyridin-2-yl)-N²-(1 S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide p-toluenesulfonate monohydrate, also called edoxaban tosylate monohydrate, or “edoxaban” herein, and has the structure:

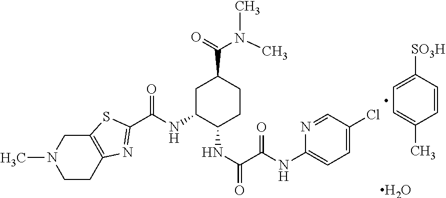

Edoxaban is orally bioavailable, as demonstrated in preclinical pharmacodynamic/pharmacokinetic (PD/PK) studies in rats and monkeys, as well as in clinical studies in human subjects. Edoxaban has been shown to be generally safe and well tolerated in doses of up to 90 mg per day. Moreover, in its capacity as an antithrombotic agent and anticoagulant, edoxaban p-toluenesulfonate monohydrate potently inhibits both free FXa and FXa complexed in prothrombinase with a subnanomolar Ki value, and its inhibitory activity is highly specific. For example, edoxaban exhibits a >10,000-fold more potent inhibition of FXa than other biologically relevant serine proteases.

Factor Xa Inhibitor Treatment of Warfarin Sensitive Subjects Identified as Having CYP2C9 and VKORC1 Polymorphisms and/or Variants of CYP2C9 and VKORC1

The methods of the present invention involve the use of FXa inhibitors, preferably direct FXa inhibitors, and preferably orally available FXa inhibitors, for subjects identified or determined to be warfarin sensitive, to provide safe and effective treatment and prevention of conditions, for example thrombosis or embolism, without excessive bleeding complications or over-anticoagulation that are associated with warfarin (or other VKA drug) use in these warfarin sensitive subjects. The subjects treated by the methods of the invention particularly include warfarin sensitive individuals identified by genetic analysis to possess genotypic polymorphisms in, or variants of, the CYP2C9 and VKORC1 genes resulting in warfarin sensitivity. In embodiments of the invention, the treatment methods involving the administration of an FXa inhibitor, such as edoxaban (administered API is edoxaban p-toluenesulfonate monohydrate), are highly suitable and advantageous for human subjects who are contraindicated for warfarin use by virtue of their carrying one or more genetic polymorphisms in CYP2C9 and/or VKORC1 alleles which result in sensitivity, especially medium or high sensitivity, to warfarin.

In accordance with the invention, a subject to be treated with an FXa inhibitor is identified or determined to be warfarin sensitive when the subject is genotyped as having one or more genetic polymorphisms in alleles of the CYP2C9 gene selected from a single nucleotide polymorphism (SNP) in the *2 allele of CYP2C9 (rs1799853), a SNP in the *3 allele of CYP2C9 (rs1057910); and/or a −1639G>A (rs9923231) SNP genetic polymorphism in the VKORC1 gene. Such genetic polymorphisms can lead to the production of functionally variant CYP2C9 and VKORC1 gene products. The level of sensitivity to warfarin can be reflected by a subject's profile of genetic polymorphism in the CYP2C9 and VKORC1 genes. In particular, a subject can be identified as being normal, sensitive, including medium sensitive or highly sensitive, to warfarin. By example and as categorized herein, the genotypic profile of a subject who is normal or not sensitive to warfarin, i.e., a “normal responder,” to warfarin therapy, is reflected by the following allelic genotypes (also termed “haplotypes”): a *1/*1 genotype in CYP2C9 and a G/G genotype in VKORC1; *1/*1 genotype in CYP2C9 and a G/A genotype in VKORC1; or a *1/*2 genotype in CYP2C9 and a G/G genotype in VKORC1. As noted supra, other CYP2C9 and VKORC1 SNP genetic polymorphisms can also be used, e.g., alone or in combination with the above allelic SNPS, to determine warfarin sensitivity in individuals undergoing testing for this purpose.

The genotypic profile of a subject who has medium sensitivity to warfarin, i.e., a “medium or moderately sensitive responder,” to warfarin therapy, is reflected by the following allelic genotypes (also termed “haplotypes”): a *1/*1 genotype in CYP2C9 and an A/A genotype in VKORC1; a *1/*2 genotype in CYP2C9 and an A/G genotype in VKORC1; a *1/*2 genotype in CYP2C9 and an A/A genotype in VKORC1; a *1/*3 genotype in CYP2C9 and a G/G genotype in VKORC1; a *1/*3 genotype in CYP2C9 and an A/G genotype in VKORC1; a *2/*2 genotype in CYP2C9 and a G/G genotype in VKORC1; a *2/*2 genotype in CYP2C9 and an A/G genotype in VKORC1; or a *2/*3 genotype in CYP2C9 and a G/G genotype in VKORC1.

The genotypic profile of a subject who has high sensitivity to warfarin, i.e., a “highly sensitive responder,” to warfarin therapy, is reflected by the following allelic genotypes (also termed “haplotypes”): a *1/*3 genotype in CYP2C9 and an A/A genotype in VKORC1; a *2/*2 genotype in CYP2C9 and an A/A genotype in VKORC1; a *2/*3 genotype in CYP2C9 and an A/G genotype in VKORC1; a *2/*3 genotype in CYP2C9 and an A/A genotype in VKORC1; a *3/*3 genotype in CYP2C9 and a G/G genotype in VKORC1; a *3/*3 genotype in CYP2C9 and an A/G genotype in VKORC1; or a *3/*3 genotype in CYP2C9 and an A/A genotype in VKORC1.

In an embodiment, warfarin sensitive subjects who are particularly suited to treatment with an FXa inhibitor, such as a direct FXa inhibitor, and indirect FXa inhibitor, or other warfarin or VKA alternative drug according to the present invention are identified as having at least a VKORC1 “A/A” genotype.

In accordance with an embodiment of the invention, an FXa inhibitor is provided for anticoagulant treatment and/or for prevention of bleeding events for a subject in need thereof, when the subject is contraindicated for treatment with warfarin. In an embodiment, such contraindication results from the subject having sensitivity to warfarin therapy. In an embodiment, such contraindication results from the subject's being identified, screened, or selected via genotyping analysis as possessing one or more SNP genetic polymorphisms in either or both of the CYP2C9 and VKORC1 genes that have been linked to sensitivity to warfarin in subjects possessing the polymorphisms. In an embodiment, the subject is genotyped as having the *2 and/or *3 alleles of the CYP2C9 gene and/or the A/A genotype of the VKORC1 gene. In an embodiment, the subject is genotyped as possessing the A/A genotype of the VKORC1 gene.

Methods and procedures for genotyping analysis, i.e., to determine specific alleles and genetic variants or polymorphisms inherited by an individual, are generally well known and practiced in the art. The mode and type of genotype analysis used for identifying, determining, or screening for genetic polymorphisms in the CYP2C9 and/or VKORC1 genes according to the present invention is not intended to be limiting. Many commercially available kits, automated processes and services are available for determining or screening genetic variations, mutations and polymorphisms in and among individuals in a population. By way of example, SNP genotyping can be carried out using nucleic acid chip formats (e.g., Fluidigm SNPtype Assays, Fluidigm, S. San Francisco, Calif.), gene chip probes (Affymetrix, Santa Clara, Calif.), PCR-based products (LCG Genomics, Beverly, Mass.), and genotype arrays/microarrays (Illumina, San Diego, Calif.). Samples for genotyping may include, without limitation, a biological fluid sample of an individual, such as blood, serum, plasma, lymph, saliva, sputum, (buccal samples), mucus, sweat, hair or hair follicle, tears, urine, amnionic fluid, bile, semen, vaginal secretions, etc., as well as stool samples, and tissue, organ and cell samples and lysates thereof. In addition, to determine warfarin sensitivity, individual genotypes can also be assessed through full genome sequencing or transcriptome sequencing. Illustratively and without limitation, following sequencing of an individual's genomic DNA or RNA, the results can be stored in an appropriate and accessible database. If the individual should ever be in need of anticoagulant therapy, a database query can be run to determine the individual's genetic sequence and the potential presence of genetic variations resulting in warfarin sensitivity, for example, in one or more polymorphisms in the CYP2C9 and/or VKORC1 genes. A finding of such genetic variation(s) would identify or characterize the individual as being warfarin sensitive, and thus, in need of an alternative warfarin treatment, such as with an FXa inhibitor, e.g., a direct FXa inhibitor such as edoxaban or an indirect FXa inhibitor, or another warfarin or VKA alternative drug or compound.

In another embodiment of the invention, individuals who carry genetic variants of the VKORC1 and CYP2C9 genes may be afflicted with, or be at risk of, over-anticoagulation or excessive bleeding following warfarin administration and comprise an optimal population of individuals who should undergo initial and sustained treatment with an FXa inhibitor pursuant to the methods of the invention, rather than with warfarin. For example, such individuals would be treated with a therapeutically effective amount of the FXa inhibitor edoxaban or other direct FXa inhibitor or warfarin or VKA alternative drug or compound. In an embodiment, an FXa inhibitor, in lieu of warfarin, is administered to individuals in need thereof, who are determined via genotyping analysis to be sensitive to warfarin treatment, particularly for those individuals who are medium or highly sensitive to warfarin and who are genotyped as carrying the *2 and/or *3 alleles of the CYP2C9 gene and/or the A/A genotype of the VKORC1 gene resulting in warfarin sensitivity.

The present invention further embraces methods of treating subjects who are sensitive to warfarin, as identified by their having genetic polymorphisms in CYP2C9 and/or VKORC1 genes associated with warfarin sensitivity, and who have or are at risk of different types of bleeding events, such as major and clinically relevant non-major (CRNM) bleeding events, with an FXa inhibitor, such as edoxaban, or a warfarin or VKA alternative. See, e.g., Example 1, Table 3 and Table 5. In an embodiment, subjects who are identified as being sensitive responders to warfarin by virtue of their having one or more variant CYP2C9 and/or VKORC1 alleles resulting in warfarin sensitivity are, in turn, more likely to experience higher levels of major bleeding events when given warfarin. In an embodiment, subjects who are identified via genotype analysis or screening, or by phenotypic analysis, as being highly sensitive responders to warfarin have a correlatively higher rate of major bleeding events than normal or medium sensitive responders to warfarin.

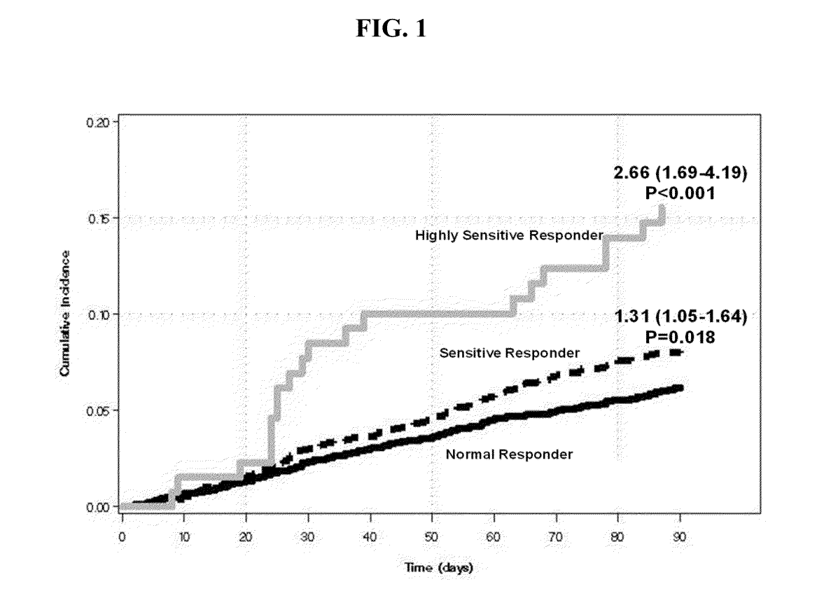

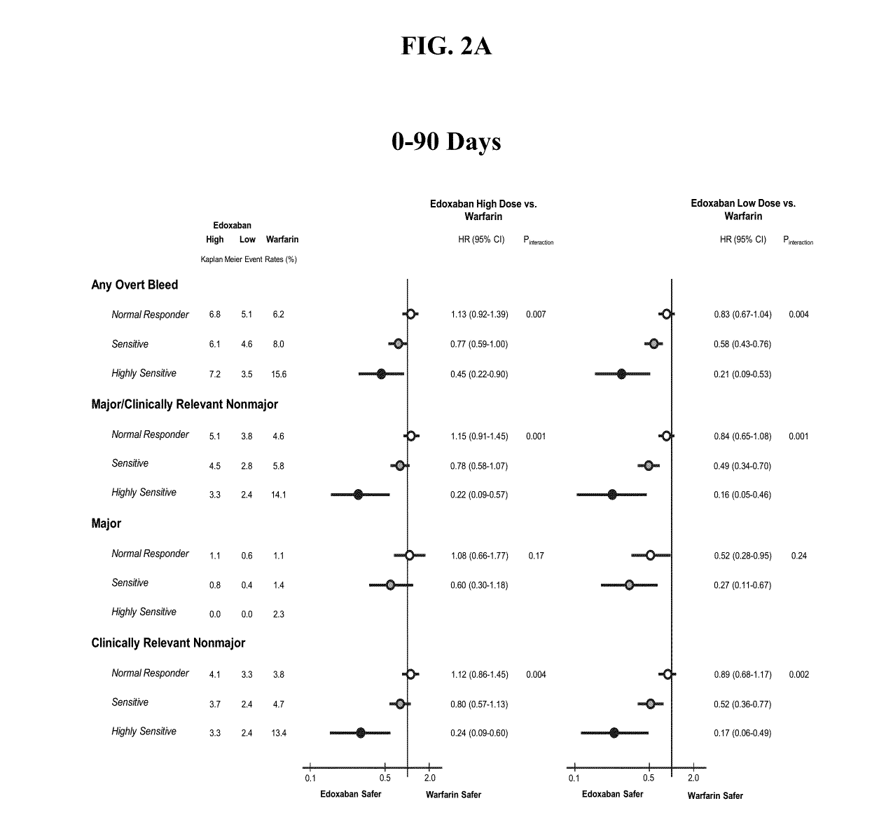

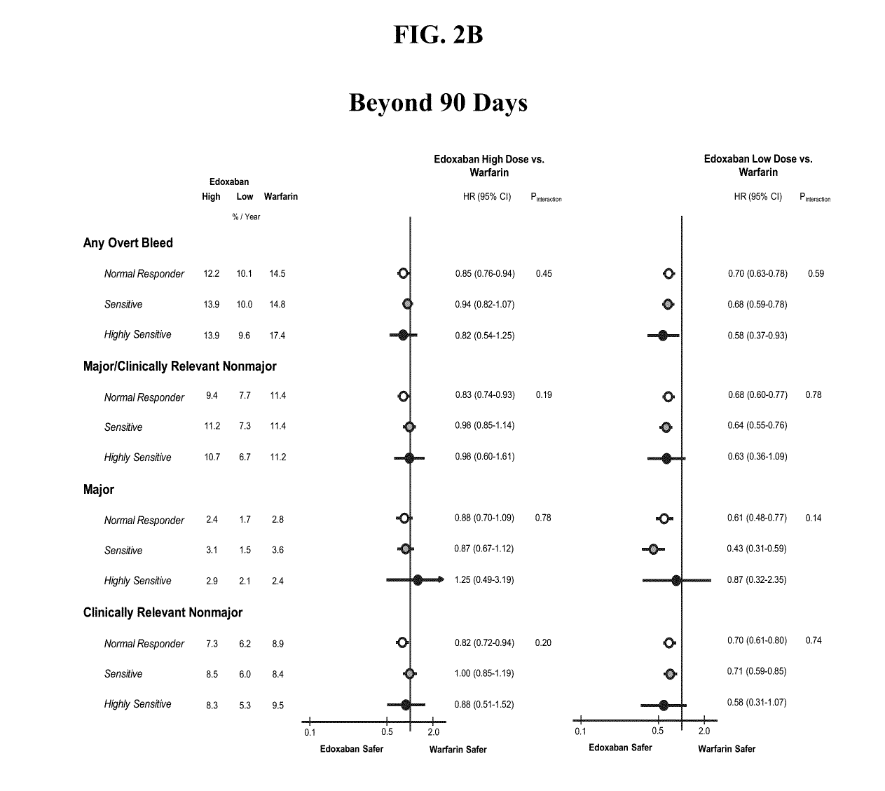

Both the medium sensitive and the highly sensitive responders to warfarin represent populations of individuals for whom treatment with an FXa inhibitor, such as edoxaban, is particularly beneficial at both lower doses, e.g., 30 mg per day, and higher doses, e.g., 60 mg per day, according to the present invention. In particular, individuals with medium and high sensitivity to warfarin as reflected by the above described genetic polymorphisms in the CYP2C9 and/or VKORC1 genes showed a higher rate of major and CRNM bleeding events in a 90 day time period after taking warfarin. In contrast, individuals identified as medium and high responders to warfarin sensitivity did not experience major bleeding or CRNM bleeding events when dosed with an FXa inhibitor, edoxaban, at doses of 30 mg per day or 60 mg per day, particularly over a 90 day time period after taking the FXa inhibitor, edoxaban. (Example 1). Thus, according to the invention, a subpopulation of subjects identified as having one or more polymorphisms in CYP2C9 and/or VKORC1 genes resulting in warfarin sensitivity exhibited more bleeding events when taking warfarin than did the same subpopulation of subjects taking an FXa inhibitor such as edoxaban. The warfarin-related bleeding events were especially evident during the first approximately 90 days of warfarin treatment by the warfarin sensitive population of subjects. A clear “gradient effect” (or gene dose effect) on bleeding risk, i.e., normal<medium sensitive<<highly sensitive to warfarin, was observed in study subjects. See, e.g., Example 1, FIGS. 1, 2A and 2B. Although the highly warfarin sensitive population of subjects was small in number (˜3.5% of the population), this population showed very high bleeding risk during warfarin therapy, based on the 3-bin genotypic analysis provided by the study, and are good candidates for treatment with an FXa inhibitor such as edoxaban according to the invention.

### Treatments and Methods Using an FXa Inhibitor for Anticoagulant Therapy

The methods of the present invention afford a newly-imparted, safe and beneficial advantage to the medical community and to warfarin sensitive patients in need of anticoagulant therapy by providing a therapeutic treatment using an FXa inhibitor for example, a direct FXa inhibitor such as edoxaban or an indirect FXa inhibitor. As discussed herein, warfarin or VKA alternative drugs or compounds are also suitable for use according to the methods of the present invention. The treatment methods described herein are particularly amenable for subjects who are determined to be warfarin sensitive and who are at increased risk of bleeding events when on warfarin; such determination can be based on genotypic and/or phenotypic assessment of the CYP2C9 and/or VKORC1 genes or gene products.

In embodiments of the methods described herein, an FXa inhibitor is preferably initially administered to a warfarin sensitive subject in need of anticoagulant treatment rather than warfarin or other VKA drug. The importance of early and/or sustained administration of an FXa inhibitor, or a warfarin or VKA alternative, was determined based on clinical studies which demonstrated a window of time (e.g., without limitation, about 90 days) during which bleeding events or over-anticoagulation rates in warfarin sensitive subjects taking warfarin were typically seen to be higher in those subjects who were identified genotypically as being medium or highly sensitive responders to warfarin sensitivity. See, e.g., Example 1 and Tables therein.

In an embodiment, the invention provides a method of preventing or reducing the risk of a major bleeding event, clinically overt bleeding event, or a clinically relevant non-major (CRNM) bleeding event in a subject in need of anticoagulant therapy and who is sensitive to warfarin as identified by having one or more genetic polymorphisms in CYP2C9 and/or VKORC1 genes, by administering to the subject a therapeutically effective amount of an FXa inhibitor or warfarin or VKA alternative. In an embodiment, the therapeutically effective amount of the FXa inhibitor or warfarin or VKA alternative is administered in a pharmaceutically acceptable composition. In an embodiment, the FXa inhibitor is a direct FXa inhibitor. In an embodiment, the direct FXa inhibitor is edoxaban or a pharmaceutically acceptable salt and/or hydrate thereof. In an embodiment, the FXa inhibitor is an indirect FXa inhibitor.

In an embodiment, the invention provides a method of determining whether an individual in need of anticoagulation therapy should be given 1) warfarin or a VKA drug, or 2) an FXa inhibitor or warfarin or VKA alternative. The method is useful in guiding or directing anticoagulant therapy by clinicians and medical practitioners. The method includes i) assaying a biological sample from the individual to identify if the individual is sensitive to warfarin. The assay can involve identifying one or more genetic polymorphisms in the CYP2C9 and/or VKORC1 genes associated with warfarin sensitivity as described herein, and/or identifying functional variants (e.g., having reduced function) of one or both of the CYP2C9 and VKORC1 gene products associated with warfarin sensitivity; ii) identifying that the individual is warfarin sensitive. The identification of warfarin sensitivity in the individual can be established by determining that the individual carries one or more genetic polymorphisms in the CYP2C9 and/or VKORC1 genes and/or that the individual has functional variants (e.g., having reduced function) of one or both of the CYP2C9 and VKORC1 gene products associated with warfarin sensitivity; iii) administering an FXa inhibitor or warfarin or VKA alternative if the individual is identified as having warfarin sensitivity according to ii). Thus, a warfarin sensitive individual is identified as carrying one or more genetic polymorphisms in the CYP2C9 and/or VKORC1 genes and/or as having functional variants (e.g., having reduced function) of one or both of the CYP2C9 and VKORC1 gene products associated with warfarin sensitivity, based on ii). Alternatively, in a step iv) of the method, if the individual is identified as not carrying one or more genetic polymorphisms in the CYP2C9 and/or VKORC1 genes and/or as not having functional variants (e.g., having reduced function) of one or both of the CYP2C9 and VKORC1 gene products associated with warfarin sensitivity based on ii), then the individual can be administered or treated with warfarin or VKA drug. It will of course be appreciated that in iv), an FXa inhibitor or a warfarin or VKA alternative can be administered to the individual instead of warfarin or VKA drug, particularly in view of the safe and effective anticoagulant treatment afforded by FXa inhibitors, such as the direct FXa inhibitor edoxaban, according to the methods of the present invention.

In an embodiment, the invention further provides a method of treating or preventing embolism, thrombus, or thromboembolism in an individual who has, or who is at risk of excessive bleeding if treated with warfarin or another VKA, by administering to the subject a therapeutically effective amount of an FXa inhibitor, or a warfarin or VKA alternative drug. In various embodiments, the individual having or at risk of having an embolism, thrombus, or thromboembolism suffers from a disease, condition, or disorder that can induce the formation of blood clots, such as those described below, by way of example. In an embodiment, the therapeutically effective amount of the FXa inhibitor or warfarin or VKA alternative is administered in a pharmaceutically acceptable composition. In an embodiment, the FXa inhibitor is a direct FXa inhibitor. In an embodiment, the direct FXa inhibitor is edoxaban or a pharmaceutically acceptable salt and/or hydrate thereof.

In embodiments of the invention, subjects who require treatment with a non-warfarin anticoagulant may suffer from various conditions, diseases, or pathologies, particularly those related to thrombotic disease or pathology. In particular, subjects, preferably human subjects, individuals, or patients, have or are at risk for thrombotic disease and conditions. Such patients may have, or be at risk for, thrombotic conditions that include, without limitation, venous thromboembolism (VTE), deep vein thrombosis (DVT), pulmonary embolism (PE), embolism, thromboembolism, and venous thrombosis (VT). DVT and PE are typically considered to be manifestations of a single pathophysiologic process that is collectively known as VTE. DVT and PE frequently present together, share the same risk factors and are associated with a high morbidity that may progress to a fatal outcome, if left untreated. While DVT is a blood clot found anywhere in the deep veins of the legs, pelvis or arms, PE occurs when part of a clot from within a deep vein detaches and embolises to the lungs, lodging in the pulmonary arteries and causing a potentially fatal condition. These subjects may be at risk of excessive bleeding or over-anticoagulation if treated with warfarin as a result of their sensitivity to warfarin and/or their carrying one or more of genetic polymorphisms in the CYP2C9 and/or VKORC1 genes as described herein.

The thrombotic conditions afflicting patients may also include peripheral arterial disease, atrial fibrillation (AF), thrombotic events following surgery, for example, but not limited to, hip replacement, knee replacement, shoulder surgery, or other orthopedic surgery. In addition, in the methods of the invention the subjects to be treated with an anticoagulant, such as an FXa inhibitor, e.g., edoxaban, may be afflicted with, or susceptible to, cerebral infarction, cerebral embolism, stroke, systemic embolism with nonvalvular atrial fibrillation, myocardial infarction, angina pectoris, pulmonary infarction, Buerger's disease, disseminated intravascular coagulation syndrome, thrombus formation after surgery, thrombus formation after valve or joint replacement, thrombus formation and reocclusion after angioplasty, systemic inflammatory response syndrome (SIRS), multiple organ dysfunction syndrome (MODS), thrombus formation during extracorporeal circulation, or blood clotting upon blood drawing. In an embodiment, the VTE may encompass PE with or without DVT, or DVT only.

In related embodiments, the methods of the invention involve administering a therapeutically effective amount of an FXa inhibitor, e.g., edoxaban or a pharmaceutically acceptable salt and/or hydrate thereof, or a pharmaceutically acceptable composition containing the FXa inhibitor such as edoxaban to a subject who is warfarin sensitive, e.g., as determined via genotypic analysis or phenotypic assay, and is in need of treatment to reduce the risk of stroke and systemic embolism associated with nonvalvular AF, for the treatment of deep vein thrombosis (DVT), for the treatment of pulmonary embolism (PE), for preventing or reducing the risk of recurrence of DVT and of PE following initial treatment for DVT and/or PE, or for prophylaxis of deep vein thrombosis following hip or knee replacement surgery. In particular embodiments, edoxaban or a pharmaceutically acceptable salt and/or hydrate thereof is administered at a dose of 60 mg taken orally once daily for the treatment of nonvalvular AF, DVT, PE and prevention of recurrent DVT and PE. A lower dose, 30 mg taken orally once daily, is typically prescribed for subjects having the foregoing pathologies who also have certain conditions or contraindications, for example, moderate to severe renal impairment, low body weight, e.g., ≦60 kg, or concomitant use of P-glycoprotein (P-gp) inhibitors, except amiodarone.

Without wishing to be limiting, treating a subject with an FXa inhibitor can involve reducing, diminishing, abrogating, ameliorating, or eliminating one or more of bleeding events, over-anticoagulation, or a disease or an adverse condition such as, for example, embolism, or thromboembolism, by the practice of the methods of the invention. In addition, the methods of the invention can reduce the risk of, or prevent the initiation of, bleeding events or over-anticoagulation in a subject in need of anticoagulant treatment who is sensitive to warfarin, and who has been screened/identified through genotype analysis or phenotypic analysis and found to carry one or more variations in the CYP2C9 and/or VKORC1 genes as described herein.

The phrase “effective amount” or “therapeutically effective amount” refers to an amount or dose sufficient to effect a desired response or outcome, to reduce, treat, ameliorate, or prevent, a symptom or condition, such as bleeding, over-anticoagulation, or a disease or disorder, e.g., embolism or thromboembolism, requiring anticoagulant treatment.

### Doses and Administration of Factor Xa Inhibitors

While human subjects and human patients are preferred, the invention contemplates the treatment of other, typical mammalian subjects, such as, for example, mice, rats, cats, dogs, horses, sheep, cows, and non-human primates. An effective amount for a particular patient may vary depending on factors such as the condition being treated, the overall health of the patient, the method, route, and dose of administration and the severity of side effects. When in combination, an effective amount is in ratio to a combination of components and the effect is not limited to individual components alone. In an aspect, an effective amount of a therapeutic such as an FXa inhibitor, or warfarin or VKA alternative will favorably modulate or affect the symptoms or condition typically by at least about 10% or more; or by at least about 20% or more; or by at least about 25% or more, or by at least about 30% or more; or preferably by at least about 50% or more, such as 60%, 70%, 80%, 90%, or 95% or more.

Preferred doses and unit dosage formulations for FXa inhibitors, for example, direct FXa inhibitors, e.g., edoxaban, are those containing an effective dose, such as provided herein for guidance, or an appropriate fraction thereof, of the active ingredient. FXa inhibitors that are small molecules may be administered at a dose of from 0.1 to 500 mg/kg per day. In various embodiments, the dose may be administered daily, twice a day, three times a day, every other day, every week, twice a week, every two weeks, every three weeks, etc., as will be appreciated by the skilled practitioner. Oral administration and/or oral dosage forms are preferred. The dose range for adult humans is generally from 5 mg to 2 g/day. Doses of FXa inhibitors may be administered as frequently or infrequently as determined by a patient's physician or medical doctor, and may be administered for a short time period, e.g., weeks, months, or for a longer time period, such as chronic administration, e.g., over several months or years. Tablets or other forms of presentation provided in discrete units may conveniently contain an amount of one or more compounds which is pharmaceutically and therapeutically effective at such dosage or as a multiple of the same, for instance, units containing 5 mg to 500 mg, usually around 10 mg to 200 mg, including discrete amounts there between, e.g., 10 mg, 15 mg, 20 mg, 30 mg, 40 mg, 50 mg, 60 mg, 70 mg, 75 mg, etc. In an embodiment, the dose is a solid dosage form. In an embodiment, the dose is an oral dose which is orally administered. In an embodiment, the FXa inhibitor, e.g., edoxaban, is administered with food. In an embodiment, the FXa inhibitor, e.g., edoxaban, is administered without food.

In a specific embodiment, the FXa inhibitor is edoxaban, and a therapeutic or effective amount of edoxaban for use in the treatment or prevention methods according to the invention, is a dose which does not cause or result in bleeding or an increased rate of bleeding following administration/dosing. In an embodiment, the effective amount of edoxaban for administration (wherein the administered API is edoxaban p-toluenesulfonate monohydrate) refers to the free base form or equivalent thereof, in which “equivalent thereof” means the same molar amount of the edoxaban free base active moiety regardless of the actual form administered.

Accordingly, the FXa inhibitor edoxaban (with dosage amounts being for the active moiety, the free base, and including the equivalent amount (e.g., the same molar amount of the free base) of any salt or hydrate or any other form thereof), may be administered in doses from 0.1 mg to at least 90 mg per day; or from 5 mg to 90 mg per day; or from 30 mg to 60 mg per day; or from 30 mg to 75 mg per day; or from 20 mg to 40 mg per day; or from 40 mg to 60 mg per day; or from 60 mg to 80 mg per day; or from 25 mg to 65 mg per day; or from 15 mg to 60 mg per day. The dose is preferably given once per day, but may also be given in multiple doses per day, for example, once, twice, three times, or four times a day. Alternatively, the dose may be given every other day or every three days, four days, or five days. Doses between the specified amounts in the ranges are also contemplated. In an embodiment of the inventive methods, the effective amount of edoxaban (as the free base) is 60 mg, or about 60 mg. In another aspect of the above methods, the effective amount of edoxaban (as the free base) is 30 mg or about 30 mg. In another aspect of the above methods, the effective amount of edoxaban (as the free base) is 15 mg or about 15 mg. In an embodiment, the dose is 60 mg, or about 60 mg, administered to a subject once per day (QD). In another embodiment, the effective amount of edoxaban in the methods of the invention is 30 mg, or about 30 mg, once per day (QD). In another embodiment, the effective amount of edoxaban in the methods of the invention is 15 mg, or about 15 mg, once per day (QD). In an embodiment, for subjects considered as fragile (e.g., the elderly; those who are health-compromised in some way, e.g., moderate renal impairment; those who are receiving drug treatments for other conditions and pathologies, e.g., those receiving P-glycoprotein inhibitors, edoxaban (as the free base) may be administered at a dose of less than 60 mg, such as 30 mg, once daily. Alternatively, the dose of edoxaban (as the free base) may be reduced by 50%, if required or desired based on an individual's response, need, or other medical considerations.

In embodiments in which the methods may involve the use of the anticoagulant rivaroxaban (XARELTO®, Janssen Pharmaceuticals, Inc. and Bayer Healthcare AG), the effective amount of the drug is 10 mg, 15 mg, or 20 mg, once or twice daily, with or without food depending on the indication and condition of the subject being treated. For example, pursuant to the rivaroxaban label, for the indication, reduction in risk of stroke in nonvalvular AE, the dose of rivaroxaban is 20 mg once daily with the evening meal if CrCl >50 mL/min, and 15 mg once daily with the evening meal if CrCl 15 to 50 mL/min. For the indication, treatment of DVT and PE, the dose of rivaroxaban is 15 mg twice daily with food for the first 21 days after which a transition of 20 mg once daily with food is made for the remainder of treatment. For the indication, reduction in risk of recurrence of DVT and PE, the dose of rivaroxaban is 20 mg once daily with food. For the indication, prophylaxis of DVT following hip or knee replacement surgery, the dose of rivaroxaban is 10 mg once daily for 35 days (hip replacement) or 10 mg once daily for 12 days (knee replacement).

In embodiments in which the methods involve the use of the anticoagulant apixaban (ELIQUIS®, Bristol-Myers Squibb Co, Princeton, N.J.), the effective amount of this drug is 2.5 mg taken orally twice daily, pursuant to the label.

In accordance with the invention, the FXa inhibitor can be administered by any route conventionally used for drug administration and as known to the skilled practitioner. By way of non-limiting example, administration may be oral, parenteral, intravenous, subcutaneous, bucal, sublabial, intranasal, intradermal, sublingual, intrathecal, intramuscular, intraperitoneal, rectal, intravaginal, gastric, or enteric. Oral administration, e.g., in single dosage form, solid dosage form, such as a tablet, or in liquid form, is preferred. In an embodiment, the FXa inhibitor is orally administered to a subject who is in need of treatment. In a particular embodiment, the FXa inhibitor edoxaban tosylate monohydrate is orally administered to a subject who is in need of treatment.

The amount of active ingredient that may be combined with a carrier material to produce a single dosage form will vary depending upon the host treated and the particular mode of administration. The precise amount of compound administered to a patient will be the responsibility of the attendant physician and route of administration. The specific dose level for any particular patient will depend upon a variety of factors, including the activity of the specific compound employed, the age, body weight, general health, sex, diet, time of administration, route of administration, rate of excretion, drug combination, the precise disorder being treated, and the severity of the disorder being treated. Also, the route of administration may vary depending on the disorder and its severity.

In other embodiments, the methods of the invention can involve combining treatment of a subject, particularly a subject who needs, or could potentially need, anticoagulant treatment or prevention with an inhibitor of FXa, in particular, edoxaban, with another anticoagulant agent, anti-thrombotic agent, and/or anti-FXa agent as secondary or adjunct treatment. Such secondary, combination, or adjunct treatments may be given prior to, concomitantly with, or subsequent to treatment with edoxaban. In some embodiments, the agents used in combination with an FXa inhibitor are heparin, thrombin/Factor IIa inhibitors (e.g., dabigatran etexilate mesylate, (PRADAXA®, Boehringer Ingelheim, Ridgefield, Conn.)), and other FXa inhibitors, e.g., direct FXa inhibitors such as rivaroxaban (Bayer Healthcare AG and Janssen Pharmaceuticals, Inc.), LY517717 (Lilly), apixaban (Bristol-Myers Squibb Company), 813893 (GlaxoSmithKline), betrixaban, AVE-3247, EMD-503982, the 3-amidoinophenylalanine-type FXa inhibitor, WX-FX4, or a pharmaceutically acceptable salt and/or hydrate thereof. In other embodiments, parenteral anticoagulant agents include, without limitation, heparin, low molecular weight heparins (e.g., dalteparin, tinzaparin, reviparin, nadroparin, ardeparin, certoparin and parnaparin), or other direct thrombin inhibitors (e.g., bivalirudin, argatroban, desirudin, lepirudin). Without limitation, other parenteral FXa inhibitors include fondaparinux.

In other embodiment, the methods of the invention can involving administering the FXa inhibitor, e.g., edoxaban, in combination with another therapeutic, drug, or bioactive agent, as desired or warranted for patient treatment. Without limitation, the therapeutic or bioactive agent can be a drug, a small molecule organic compound, or a biologic that may be co-administered with the FXa inhibitor, either at the same time or at different times. Therapeutic or biologically active agents can be of numerous classes, illustratively, antibiotics, antimicrobial agents, antidepressant agents, anti-anxiety agents, anti-asthmatic agents, anti-emetic agents, anti-diabetic agents, anti-fungal agents, anti-hypertension agents, anti-inflammatory agents, immunosuppressive agents, anti-immunosuppressive agents, anti-neoplastic agents, anti-impotence agents, anti-viral agents, anti-HIV agents, anxiolytic agents, fertility or contraceptive agents, antithrombotic agents, prothrombotic agents, hormones, vaccines, vitamins, and the like. Additional examples of such agents can be found, e.g., in Goodman & Gilman's, 2011, The Pharmacological Basis of Therapeutics, Twelfth Edition, L. Brunton, B. Chabner, B. Knollman, eds., McGraw-Hill. More particularly, other drugs that may be used with an FXa inhibitor, e.g., edoxaban, include statins, e.g., atorvastatin; P-gp substrates, e.g., digoxin; antiplatelets; antithrombotic agents; fibrinolytics; non-steroidal anti-inflammatory drugs, e.g., acetylsalicylic acid (aspirin); naproxen; and proton pump inhibitors (PPIs), e.g., esomeprazole.

In its embodiments, the invention provides methods conducive to improving treatments and treatment options for individuals afflicted with, or at risk of, embolism, thromboembolism, or thrombosis, wherein the individuals can particularly benefit from treatment or therapy with edoxaban. Thus, the term “treating” in a general sense as used herein refers to preventing, inhibiting, curing, reversing, attenuating, alleviating, abrogating, minimizing, suppressing, reducing, decreasing, or eliminating the deleterious effects of a disease state, disease progression, a disease causative agent, or other abnormal condition, such as bleeding or over-anticoagulation. For example, treatment may involve alleviating a symptom, although not necessarily all of the symptoms of a disease, or attenuating the progression of a disease. Treatment can embrace partially or totally reducing, abrogating, delaying, reversing, reducing, eliminating, or preventing embolism, thromboembolism, thrombosis, and related disorders, or preventing the onset, development, or recurrence of such symptoms, conditions, or disorders in a mammal, particularly a human.

As will be appreciated by the skilled practitioner, the FXa inhibitor, e.g., edoxaban, pursuant to the invention is preferably used in a therapeutically effective amount, which is intended to qualify as the amount or dose of the treatment, e.g., drug, compound, active ingredient, composition, or agent, determined or necessary to treat or prevent bleeding events, over-anticoagulation, embolism, thrombosis, or thromboembolisms in a therapeutic or treatment regimen. This includes combination therapy involving the use of multiple therapeutic agents, such as a combined amount of a first and second treatment, in which the combined amount will achieve the desired biological treatment response as described herein above. A “therapeutically effective amount” refers to the amount of a drug or compound that, when administered, is sufficient to prevent the development of, or reduce, alleviate, attenuate, or abrogate to some extent, one or more of the symptoms of the disorder being treated. The term “therapeutically effective amount” also refers to the amount of a drug or compound that is sufficient to elicit the biological or medical response of a cell, tissue, system, animal, or human that is being sought by a practitioner, e.g., a medical doctor, clinician, veterinarian, or researcher.

The invention also embraces the administration of an FXa inhibitor in a pharmaceutically or therapeutically acceptable composition, wherein the composition comprises a pharmaceutically or therapeutically effective amount of the FXa inhibitor, e.g., edoxaban. As will be understood by the skilled practitioner, such compositions may comprise pharmaceutically acceptable carriers, excipients, diluents, or vehicles, e.g., buffered saline, which are suitable for use in subjects without incompatibility, instability, undue toxicity, allergic response, and the like. Other pharmaceutically acceptable ingredients are also suitable for use in the compositions, for example and without limitation, emulsifiers, antioxidants, antimicrobials, preservatives, thickeners, stabilizers, humectants, vitamins, minerals, and the like, as conventionally known and used in the pharmaceutical arts.

The present invention provides a new, desirable and safe treatment modality for reducing the risk of, or reducing the risk of recurrence of, a bleeding event in a subject, preferably a human subject, who is determined to be warfarin sensitive, for example, via genotypic analysis, and having one or more of the genetic polymorphisms in CYP2C9 and VKORC1 as described herein, wherein the subject is afflicted with, at risk of, or at risk for recurrence of, embolism, thrombosis, or thromboembolism, or another condition requiring anticoagulant therapy, e.g., oral anticoagulant therapy. Such treatment involves the administration of an inhibitor of FXa, e.g., a direct FXa inhibitor, such as edoxaban or a pharmaceutically acceptable salt and/or hydrate thereof. In accordance with the invention, FXa inhibitor treatment was surprisingly and advantageously found to significantly reduce bleeding events, relative to warfarin treatment, in subjects undergoing treatment with the FXa inhibitor for a thrombotic condition, such as AF, particularly for those subjects carrying one or more genetic polymorphisms in genes CYP2C9 and VKORC1 resulting in medium to high sensitivity to warfarin. An exemplary and highly beneficial FXa inhibitor in accordance with an embodiment of the invention and as described herein is edoxaban or a pharmaceutically acceptable salt and/or hydrate thereof.

The relative safety and efficacy of the treatment methods of the invention are exemplified below. In general, patients with a genetic predisposition to warfarin sensitivity are more likely to be over-anticoagulated and suffer higher rates of bleeding events when taking warfarin. In these individuals, the relative safety and effect of an FXa inhibitor such as edoxaban compared with warfarin were observed, especially in the early time period, e.g., without limitation, about 90 days, of warfarin administration. Genotypic polymorphisms in one or both of the CYP2C9 and VKORC1 genes, namely, those correlated with or identified as conferring medium or high warfarin sensitivity on carriers of such CYP2C9 and/or VKORC1 polymorphisms are particularly amenable to safe and effective treatment with an FXa inhibitor such as edoxaban to treat and prevent bleeding events or over-anticoagulation in patients in need thereof.

### EXAMPLES

### Example 1

This Example describes the clinical study and results thereof in which treatment with the direct FXa inhibitor edoxaban at both low and high doses was unexpectedly discovered to reduce or decrease significantly the incidence of bleeding in patients who had been genotyped as sensitive or highly sensitive responders to warfarin, as compared to warfarin treatment, particularly within a determined time frame.

### INTRODUCTION

Polymorphisms in the CYP2C9 gene (which encodes an enzyme responsible for the metabolism of the more active S-warfarin isomer) and the VKORC1 gene (which encodes vitamin-K epoxide reductase, the molecular target of warfarin) affect an individual's sensitivity to warfarin and account for approximately 40% of the variability in the response. (Johnson, J. A. et al., 2011, Clin Pharmacol Ther, 90:625-6291). Based on these observations, the FDA has indicated that an individual's CYP2C9 and VKORC1 genotype information, when available, can assist in dose selection. (http//www.accessdata.fda.gov/drugsatfda_docs/label/2010/009218s1081b1.pdf). To date, however, the potential links among these polymorphisms and clinical outcomes, and adverse events involving bleeding and over-anticoagulation, have not been fully elucidated and may even have been frequently under-reported.

Given the limitations of warfarin, novel oral anticoagulants, such as the FXa inhibitor edoxaban, provide more predictable pharmacokinetics than warfarin. The ENGAGE AF-TIMI 48 trial compared edoxaban to warfarin in patients with atrial fibrillation (AF) followed for a median of 2.8 years. A pre-specified genetic study was also included within the ENGAGE AF-TIMI 48 trial. Specifically, the hypotheses tested were that (1) among patients treated with warfarin, those genetically predisposed to be more sensitive to warfarin would have higher rates of bleeding compared with normal responders, and (2) edoxaban would consequently compare particularly favorably with warfarin in such patients.

### Brief Description of Study

The ENGAGE AF-TIMI 48 study was a randomized, double-blind trial comparing warfarin to two doses of the FXa inhibitor edoxaban in patients with AF, (median follow-up, 2.8 years). The pre-specified genetic analysis included 14,348 participants, who were genotyped for functional genetic variants in CYP2C9 and VKORC1. The relationships among genotype, pharmacologic response, and clinical outcomes during oral anticoagulant therapy were tested.

### Patient Population

The ENGAGE AF-TIMI 48 trial enrolled 21,105 patients who were age ≧21 years with AF documented on an electrical tracing within 12 months, a CHADS₂ risk score ≧2, and anticoagulation planned for the trial duration (Gage, B. F. et al., 2001, JAMA, 285:2864-2870; Ruff, C. T. et al., 2010, American Heart Journal, 160:635-641; Giugliano, R. P. et al., N Engl J Med, 369:2093-2104). In this multicenter, double-blind, double-dummy, three-arm trial, patients were randomly assigned (1:1:1) to receive warfarin, edoxaban high-dose, or edoxaban low-dose. The starting warfarin dosage was determined by the local investigator based on the clinical profile of the subject, with the use of a computer-based algorithm (e.g., www.warfarindosing.org) recommended. The protocol stipulated specific visits for dose titration, and warfarin was dose-adjusted to an international normalized ratio (INR) of 2.0 to 3.0. The INR values were measured using an encrypted point-of-care device. To maintain blinding, sham INR values were generated for patients who were randomly assigned to edoxaban. The high-dose edoxaban group received 60 mg daily and the low-dose group 30 mg daily, with a dose reduction by half based on renal function, weight, and concomitant use of potent P-glycoprotein inhibitors.

For warfarin dosing, the protocol indicated that the starting dosage (mg/day) of warfarin (or placebo-to-match) should be determined by the local investigator based on the clinical profile of the subject; the use of a computer based algorithm (e.g., www.warfarindosing.org) was recommended. In the first month of treatment, visits occurred at Days 8, 15, and 29, and more frequently if indicated. Thereafter, INRs were measured at least monthly using an encrypted point-of-care device. To maintain blinding, sham INR values were provided for patients randomized to edoxaban.

For edoxaban dosing, the edoxaban dose was reduced by half if any of the following were present at randomization or during the course of the study: CrCl 30-50 ml/min, body-weight ≦60 kg, or concomitant verapamil or quinidine (both are potent P-glycoprotein inhibitors).

During follow-up, safety and efficacy were recorded. A clinical endpoint committee, unaware of study treatment, adjudicated all deaths and suspected cases of bleeding, cerebrovascular events, systemic embolic event (SEE), and myocardial infarction (MI). Any overt bleeding was defined as ISTH major, clinically relevant non-major, and minor bleeding.

For bleeding events, a “major bleeding event” is defined as a clinically overt bleeding event (i.e., bleeding that is visualized by examination or radiologic imaging) that meets ≧1 of the following: 1. fatal bleeding; 2. Symptomatic bleeding in a critical area or organ, such as retroperitoneal, intracranial, intraocular, intraspinal, intraarticular, pericardial, or intramuscular with compartment syndrome; 3. A clinically overt bleeding event that causes a fall in hemoglobin level of ≧2.0 g/dL (1.24 mMol/L), adjusted for transfusions. (e.g., Tables 3 and 5). Each 1 unit of packed red blood cell or whole blood is counted as a 1.0-g/dL decrease in hemoglobin. In the case of surgical procedural-related bleeding, the bleeding must be in excess of that normally associated with the surgery/procedure. In the absence of hemoglobin data, a fall of hematocrit of ≧6.0%, adjusted for transfusion, will satisfy the criteria for a major bleeding event.

Major bleeding events may also be further sub-classified as life threatening or non-life threatening. A “life-threatening major bleed” is defined as a bleeding event that either is intracranial or is associated with hemodynamic compromise requiring intervention. A “clinically relevant nonmajor bleeding event” is defined as a clinically overt bleeding event that requires medical attention. Examples of bleeding requiring medical attention include, but are not limited to, bleeding events that result in the following diagnostic or therapeutic measures: requires or prolongs hospitalization; laboratory evaluation; imaging studies; endoscopy; colonoscopy; cystoscopy; or bronchoscopy; nasal packing; compression; ultrasound-guided closure of an aneurysm; coil embolization; inotropic support; surgery; interruption or stopping study medication at the advice of a health care provider; or changing concomitant therapies (e.g., reducing the dose of or discontinuing aspirin) at the advice of a health care provider. An outpatient visit without any of the above or similar diagnostic/therapeutic measures does not satisfy the criteria for “requiring medical attention”.

Other overt bleeding events that do not fulfill the criteria of a major bleeding event or a clinically relevant non-major bleeding event (e.g., epistaxis that does not require medical attention) was classified as “minor bleeding events”. All other events (e.g., decline in hemoglobin with no overt bleeding event) were classified as “no bleeding events.”

### Genotypes

Genotypes were determined for CYP2C9 (*2 and *3 alleles; rs1799853 and rs1057910) and VKORC1 (−1639G>A; rs9923231) utilizing the Sequenom genotyping methodology performed by ILS Genomics (Morrisville, N.C.).

### SNP Genotypes in CYP2C9 and VKORC1

The RefSeq SNP, rs1799853, is a SNP in the CYP2C9 gene and is linked to poor warfarin metabolism. The rs1799853(T) allele encodes a variant amino acid, cysteine, which has been linked to poor metabolism of warfarin and thus sensitivity to this drug. The common nomenclature for this polymorphism is CYP2C9*2 (Cys amino acid, T allele; the SNP is also known as C430T or Cys144Arg). Warfarin dosage is monitored through an INR (International Normalized Ratio) and proper dosing is influenced by a variety of factors including warfarin metabolism and diet. Predicting the effect of CYP2C9 variants on warfarin drug metabolism can also involve consideration of CYP2C9*3, defined as the common loss of function or reduction in function variant rs1057910(C). Individuals carrying this SNP may also show an increased risk of developing acute gastrointestinal bleeding during the use of nonsteroidal anti-inflammatory drugs (NSAIDs) that are CYP2C8 or CYP2C9 substrates, such as aceclofenac, celecoxib, diclofenac, ibuprofen, indomethazine, lornoxicam, meloxicam, naproxen, piroxicam, tenoxicam and valdecoxib.

Several SNPs in the VKORC1 gene have been linked to warfarin sensitivity, the most common of which is the RefSeq SNP, rs9923231. The orientation of the SNP is often published as being on the opposite strand compared to the orientation in dbSNP; thus, this SNP is also identified as a G>T. In addition, rs9923231 is also known as −1639G>A with the minus indicating that it is in an upstream promoter; 3673 based on its position in GenBank accession number AY587020, as well as VKORC1*2. Generally, patients who are carriers of the rs9923231(T) allele SNP and having a condition such as venous thromboembolism (VTE) require significantly reduced doses of warfarin and are otherwise at a higher risk of serious bleeding.

Clinical studies have demonstrated that the rs9923231(A) SNP, and the tightly linked intron 1 SNP rs9934438(T), predict warfarin dose more accurately than does the intron 2 SNP 1542G>C in African American individuals. An increased warfarin dose requirement in African Americans was accounted for by a lower frequency of the rs9923231(T) allele in this ethnic population. The T allele at rs9923231 is a suitable biomarker for warfarin dosing across ethnic populations.

In the ENGAGE AF-TIMI 48 trial, based on a combination of variants, patients were grouped into functional genetic “bins” for analysis pursuant to the FDA changes to the warfarin label. (http//www.accessdata.fda.gov/drugsatfda_docs/label/2010/009218s1081b1.pdf). The three genetic bins included normal responders, sensitive responders, and highly sensitive responders to warfarin dosing (See, Table 1). In brief, the “normal responders” embraced patients who were genotyped as “*1/*1 and *1/*2 for CYP2C9, and G/G and A/G for VKORC1, representing about 62% of the patients; the “medium (or moderate) sensitivity responders” embraced individuals who were genotyped as *1/*3, *2/*2, or *2/*3 for CYP2C9, and A/A for VKORC1, representing about 34.5% of the patients; and the “high sensitivity responders” embraced individuals who were genotyped as *3/*3 for CYP2C9, and A/A for VKORC1, representing about 3.5% of the patients in the trial.

### Statistical Analysis

Among the warfarin treated patients, baseline characteristics, time to first therapeutic INR, time-in-therapeutic range (TTR), and final average warfarin dose were compared among normal, sensitive, and highly sensitive responders. Time to therapeutic INR is defined as the first INR value between 2.0 and 3.0. Of the 3,877 warfarin treated subjects who were genotyped for CYP2C9 and VKORC1 polymorphisms and categorized into genotype sets or “bins”, the median time to therapeutic range was 9.0 days for genetically defined normal responders compared to 7.0 days for sensitive responders.

The time-in-therapeutic range (TTR) in the warfarin group was calculated by linear interpolation (Rosendaal, F. R., 1993, Thrombosis and Haemostasis, 69:236-239), rounding interpolated values to the nearest 0.1 (Verhovsek, et al., 2008, BMC Geriatrics, 8:13). Cox proportional hazards models were used to compare the bleeding and efficacy outcomes among patients using the normal responders as the referent group. Any overt bleeding was evaluated with subcategories of bleeding tested for directional consistency based on hazard ratios and 95% confidence intervals. Efficacy analyses included ischemic stroke and SEE, in addition to mortality. Based on prior studies, analyses were conducted from baseline to day 90 and beyond 90 days (Anderson, J. L. et al., 2012, Circulation, 125:1997-2005; Pirmohamed, M. et al., 2013, N Engl J Med, 369:2294-2303; Verhoef, T. I. et al., 2013, N Engl J Med, 369:2304-2312).

Analyses were conducted among randomized patients with a genetic sample (N=14,348) who received at least one dose of study drug and incorporated an “on-treatment” period, defined as the time between first study-drug dose and the earlier of three days post last dose or end of the treatment period. In a sensitivity analysis, safety and efficacy analyses were adjusted for covariates that differed across genotype bins (race, region, creatinine clearance, and weight) and previous exposure to a VKA. The safety and efficacy of each edoxaban dosing regimen relative to warfarin were compared in patients stratified by genotype bin. Interaction terms were generated using Cox proportional hazards models for each regimen compared with warfarin.

TABLE 1
Average Warfarin Dose (mg) Among Warfarin Treated Patients Across Genotype Bins

| | CYP2C9 | | | | | | | | | | | |
|---|---|---|---|---|---|---|---|---|---|---|---|---|
| | *1/*1 | | *1/*2 | | *1/*3 | | *2/*2 | | *2/*3 | | *3/*3 | |
| | G/G | | | | | | | | | | | |
| | | | | | Medium | | Medium | | Medium | | Highly | |
| | Normal | | Normal | | Sensitive | | Sensitive | | Sensitive | | Sensitive | |
| VKORC1 | Responder | | Responder | | Responder | | Responder | | Responder | | Responder | |
| | MEAN | 5.9 | MEAN | 4.9 | MEAN | 4.2 | MEAN | 3.6 | MEAN | 3.2 | MEAN | 1.5 |
| | STD | 2.2 | STD | 1.9 | STD | 1.8 | STD | 1.4 | STD | 1.0 | STD | 0.7 |
| | N | 1146 | N | 333 | N | 200 | N | 30 | N | 18 | N | 6 |
| | G/A | | | | | | | | | | | |
| | | | Medium | | Medium | | Medium | | Highly | | Highly | |
| | Normal | | Sensitive | | Sensitive | | Sensitive | | Sensitive | | Sensitive | |
| | Responder | | Responder | | Responder | | Responder | | Responder | | Responder | |
| | MEAN | 4.4 | MEAN | 3.7 | MEAN | 3.2 | MEAN | 2.7 | MEAN | 2.0 | MEAN | 1.2 |
| | STD | 1.8 | STD | 1.5 | STD | 1.3 | STD | 1.0 | STD | 1.2 | STD | 0.5 |
| | N | 1463 | N | 400 | N | 263 | N | 24 | N | 27 | N | 9 |
| | A/A | | | | | | | | | | | |
| | Medium | | Medium | | Highly | | Highly | | Highly | | Highly | |
| | Sensitive | | Sensitive | | Sensitive | | Sensitive | | Sensitive | | Sensitive | |
| | Responder | | Responder | | Responder | | Responder | | Responder | | Responder | |
| | MEAN | 2.9 | MEAN | 2.4 | MEAN | 2.0 | MEAN | 1.5 | MEAN | 1.4 | MEAN | 1.0 |
| | STD | 1.3 | STD | 0.8 | STD | 0.8 | STD | 0.8 | STD | 0.5 | STD | 0.0 |
| | N | 639 | N | 119 | N | 73 | N | 8 | N | 12 | N | 3 |

### Results

The genotype acquisition rates were 99.99%. For VKORC1, CYP2C9*2, and CYP2C9*3, observed allele frequencies were similar to previously published values in each major ethnic group (Limdi, N. A. et al., 2008, Pharmacotherapy, 28(9):1084-97), and all three genes were in Hardy-Weinberg Equilibrium. Based on the combination of their CYP2C9 and VKORC1 genotypes, among warfarin treated subjects, 61.7% were normal responders to warfarin, 35.4% were sensitive responders to warfarin, and 2.9% were highly sensitive responders to warfarin.

Age, sex, qualifying risk factors, CHADS₂ score, and type of atrial fibrillation were similar across genotype, while race, creatinine clearance, weight, and previous exposure to a vitamin K antagonist differed across genotype (Table 4). Based on knowledge in the art, the genotype differences in different racial groups would be expected in view of the differences in allelic frequencies in VKORC1 across the different racial groups.

Pharmacologic Outcomes among Warfarin Treated Patients

The time to first therapeutic INR varied across genotype bins and was the longest in normal responders and the shortest in highly sensitive responders (Table 2). The time in therapeutic range differed across genotype bins, with the differences most pronounced at the earlier time points and attenuated in analyses conducted beyond 90 days (Table 2). Notably, the median [interquartile range] proportion of time patients were over-anticoagulated with an INR >3 through 90 days was 2.2% [0.0, 20.2], 8.4% [0.0, 25.8], and 18.3% [0.0, 32.6] for normal, sensitive, and highly sensitive responders (P<0.001); conversely, the proportion of time patients were under-anticoagulated with an INR <2 through 90 days was 30.3% [12.4, 56.2], 23.8% [9.3, 46.1], and 23.0% [9.0, 41.6] across the three groups (P<0.001). In terms of dosing, the mean±SD final warfarin dose was 5.1±2.1, 3.3±1.5, and 1.8±0.9 mg per day for normal, sensitive, and highly sensitive responders, and the full dosing information across each of the genotype combinations is presented in Table 1.

### Clinical Outcomes by Genotype

Among the warfarin treated patients, during the first 90 days, a total of 334 patients had an overt bleeding event. Sensitive, particularly medium sensitive responders and highly sensitive responders experienced higher rates of bleeding as compared with normal responders (HR 1.31, 95% CI 1.05-1.64, P=0.018 and HR 2.66, 95% CI 1.69-4.19, P<0.001, respectively; FIG. 1). There were directionally consistent results seen with major bleeding, clinically relevant non-major bleeding, and intracranial hemorrhage (Table 3). In Table 3, “KM” refers to Kaplan Meier curve or estimator (KM curve or KM estimator), which, as known in the art, is a statistical modeling technique to estimate survival of subjects (or other key event) over time; i.e., an ‘event rate.’ Adjusting these analyses for clinical covariates, including previous exposure to a VKA, yielded similar findings (Table 5).

TABLE 2
INR and Dosing Data Among Warfarin Treated Patients AcrossGenotype Bins

| | | | Highly | |
|---|---|---|---|---|
| | Normal | Sensitive | Sensitive | |
| | Responder | Responder | Responder | |
| Characteristic | (N = 2982) | (N = 1711) | (N = 140) | P Value |
| Mean (SD) and | 21.5 (50.7) | 12.7 (30.2) | 8.5 (10.7) | <0.001 |
| median time to | 9.0 [1.0, 921.0] | 7.0 [1.0, 683.0] | 5.5 [1.0, 83.0] | |
| therapeutic INR | | | | |
| [interquartile range]- | | | | |
| days | | | | |
| Time to therapeutic | | | | |
| INR-n (percent) | | | | |
| <1 week | 1098 (37.8) | 815 (48.4) | 76 (55.1) | <0.001 |
| ≧1 week and <2 | 753 (26.0) | 491 (29.2) | 37 (26.8) | |
| weeks | | | | |
| ≧2 weeks and <3 | 348 (12.0) | 156 (9.3) | 13 (9.4) | |
| weeks | | | | |
| ≧3 weeks and <4 | 193 (6.7) | 81 (4.8) | 5 (3.6) | |
| weeks | | | | |
| ≧4 weeks | 509 (17.5) | 141 (8.4) | 7 (5.1) | |
| Mean (SD) and | 48.9 (35.8) | 36.6 (32.3) | 28.1 (28.9) | <0.001 |
| median time | 44.4 [18.5, 85.2] | 29.6 [7.4, 57.1] | 18.5 [3.7, 41.2] | |
| [interquartile range] | | | | |
| in INR ranges | | | | |
| through 28 days- | | | | |
| percent | | | | |
| <2 | 41.6 (32.5) | 47.5 (30.9) | 41.9 (29.3) | <0.001 |
| 2-3 | 40.7 [11.1, 66.7] | 44.4 [22.2, 70.4] | 33.3 [18.5, 63.0] | |
| >3 | 9.5 (18.5) | 15.9 (23.9) | 30.1 (30.9) | <0.001 |
| | 0 [0, 11.1] | 0 [0, 29.6] | 23.8 [0, 58.8] | |
| Mean (SD) and | 37.3 (29.7) | 30.8 (26.4) | 27.8 (24) | <0.001 |
| median time | 30.3 [12.4, 56.2] | 23.8 [9.3, 46.1] | 23.0 [9.0, 41.6] | |
| [interquartile range] | | | | |
| in INR ranges | | | | |
| through 90 days- | | | | |
| percent | | | | |
| <2 | 50.5 (27.9) | 54.0 (26.3) | 51.3 (25.5) | <0.001 |
| 2-3 | 51.7 [29.2, 72.3] | 53.9 [34.8, 75.3] | 51.7 [29.4, 68.5] | |
| >3 | 12.1 (17.2) | 15.2 (18.4) | 20.9 (19.3) | <0.001 |
| | 2.2 [0, 20.2] | 8.4 [0, 25.8] | 18.3 [0, 32.6] | |
| Mean (SD) and | 23.5 (19.1) | 21.1 (17.1) | 21.0 (17.2) | <0.001 |
| median time | 19.1 [11.4, 29.4] | 17.1 [10.4, 26.3] | 18.5 [9.7, 26.1] | |
| [interquartile range] | | | | |
| in INR ranges beyond | | | | |
| 90 days-percent | | | | |
| <2 | 63.2 (19.0) | 65.0 (17.9) | 63.8 (18.3) | 0.030 |
| 2-3 | 66.2 [55.1, 75.8] | 67.6 [56.1, 76.7] | 66.6 [56.6, 73.4] | |
| >3 | 13.3 (11.1) | 13.9 (11.5) | 15.2 (12.2) | 0.051 |
| | 11.7 [6.4, 17.7] | 12.2 [6.6, 18.9] | 13.7 [6.9, 20.7] | |
| Mean (SD) final | 5.1 (2.1) | 3.3 (1.5) | 1.8 (0.9) | <0.001 |
| average warfarin dose | | | | |
| per day-mg | | | | |

TABLE 3
Safety Outcomes Among Warfarin Treated Patients Across Genotype Bins

| | 0-90 Days | | | | | | | | | |
|---|---|---|---|---|---|---|---|---|---|---|
| | | | | | | | Highly | | Highly | |
| | Normal | | Sensitive | | Sensitive | | Sensitive | | Sensitive | |
| | Responder | | Responder | | vs. | | Responder | | vs. | |
| | (N = 2982) | | (N = 1717) | | Normal | | (N = 143) | | Normal | |
| | | KM | | KM | | 95% | | KM | | 95% |
| | n | Rates | n | Rates | HR | CI | n | Rates | HR | CI |
| Any overt bleed | 179 | 6.2 | 134 | 8.0% | 1.31 | (1.05- | 21 | 15.6 | 2.66 | (1.69- |
| | | | | | | 1.64) | | | | (4.19) |
| Major/Clini- | 133 | 4.6 | 96 | 5.8% | 1.26 | (0.97- | 19 | 14.1 | 3.21 | (199- |
| cally relevant | | | | | | 1.64) | | | | 5.18) |
| non-major | | | | | | | | | | |
| bleed | | | | | | | | | | |
| Major bleed | 31 | 1.1 | 23 | 1.4% | 1.29 | (0.75- | 3 | 2.3 | 2.12 | (0.65- |
| | | | | | | 2.21) | | | | 6.92) |
| Clinically | 109 | 3.8 | 78 | 4.7% | 1.25 | (0.93- | 18 | 13.4 | 3.69 | (2.25- |
| relevant non- | | | | | | 1.67) | | | | 6.06) |
| major bleed | | | | | | | | | | |
| Intracranial | 6 | 0.2 | 4 | 0.2% | 1.16 | (0.33- | 1 | 0.8 | 3.63 | (0.44- |
| bleed | | | | | | 4.10) | | | | 30.02) |
| Life-threatening | 7 | 0.2 | 3 | 0.2% | 0.74 | (0.19- | 1 | 0.8 | 3.10 | (0.38- |
| bleed | | | | | | 2.87) | | | | 25.17) |
| | Beyond 90 Days | | | | | | | | | |
| | | | | | | | Highly | | Highly | |
| | Normal | | Sensitive | | Sensitive | | Sensitive | | Sensitive | |
| | Responder | | Responder | | vs. | | Responder | | vs. | |
| | (N = 2803) | | (N = 1616) | | Normal | | (N = 129) | | Normal | |
| | | %/ | | %/ | | 95% | | %/ | | 95% |
| | n | yr | n | yr | HR | CI | n | yr | HR | CI |
| Any overt bleed | 777 | 14.5 | 459 | 14.8 | 1.02 | (0.91- | 41 | 17.4 | 1.20 | (0.88- |
| | | | | | | 1.15) | | | | 1.64) |
| Major/Clini- | 637 | 11.4 | 371 | 11.4 | 1.00 | (0.88- | 28 | 11.2 | 0.98 | (0.67- |
| cally relevant | | | | | | 1.14) | | | | 1.44) |
| non-major | | | | | | | | | | |
| bleed | | | | | | | | | | |
| Major bleed | 174 | 2.8 | 128 | 3.6 | 1.28 | (1.02- | 7 | 2.4 | 0.86 | (0.41- |
| | | | | | | 1.61) | | | | 1.80) |
| Clinically | 508 | 8.9 | 279 | 8.4 | 0.94 | (0.82- | 24 | 9.5 | 1.06 | (0.70- |
| relevant non- | | | | | | 1.09) | | | | 1.62) |
| major bleed | | | | | | | | | | |
| Intracranial | 36 | 0.6 | 26 | 0.7 | 1.25 | (0.75- | 2 | 0.7 | 1.20 | (0.29- |
| bleed | | | | | | 2.07) | | | | 4.94) |
| Life-threatening | 27 | 0.4 | 34 | 0.9 | 2.18 | (1.32- | 3 | 1.0 | 2.39 | (0.73 |
| bleed | | | | | | 3.62) | | | | 7.80) |

Beyond 90 days, genotype was not associated with an increased risk of any overt bleeding, but was associated with an increased risk of major and life-threatening bleeding, albeit with wide confidence intervals in the highly sensitive responders (Table 3). In terms of efficacy, within this analysis, a total of 16 patients had an ischemic stroke or SEE event, and a total of 12 patients died in the first 90 days. There were no significant associations between genotype and any of these outcomes (Table 6).

### Relative Safety of Edoxaban Versus Warfarin by Genotype

In the trial overall, the HRs (95% CI) for any overt bleeding with high-dose edoxaban vs. warfarin and low-dose edoxaban vs. warfarin were 0.87 (95% CI 0.82-0.92) and 0.66 (95% CI 0.62-0.71). During the first 90 days, when comparing edoxaban high-dose vs. warfarin among normal responders, sensitive responders, and highly sensitive responders, the HRs for bleeding were 1.13 (0.92-1.39), 0.77 (0.59-1.00), and 0.45 (0.22-0.90) (P_{interaction}=0.007, FIG. 2A). Similarly, the HRs for bleeding with edoxaban low-dose versus warfarin among these three groups were 0.83 (0.67-1.04), 0.58 (0.43-0.76), and 0.21 (0.09-0.53) (P_{interaction}=0.004, FIG. 2A). Data for the individual bleeding outcomes were directionally consistent. Comparisons for intracranial hemorrhages and life threatening bleeds were limited due to the small number of events (Table 7). Beyond 90 days, the overall HRs for bleeding with high-dose edoxaban versus warfarin and low-dose edoxaban versus warfarin were 0.88 (95% CI 0.81-0.95) and 0.69 (95% CI 0.63-0.75), and there was no significant interaction between genotype and bleeding with edoxaban versus warfarin (FIG. 2B).

### CONCLUSION

The results of the large, prespecified pharmacogenetic study described in this Example demonstrate that genetic polymorphisms in CYP2C9 and VKORC1 genes affect not only the pharmacologic but also the clinical response to warfarin. Specifically, based on the FDA-recommended combination of CYP2C9 and VKORC1 genotypes, among warfarin treated subjects, 61.7% were normal responders to warfarin, 35.4% were sensitive responders, and 2.9% were highly sensitive responders. As compared with normal responders, sensitive and highly sensitive responders spent a greater proportion of time over-anticoagulated, with an INR >3, through the first 90 days (2.2%, 8.4%, and 18.3% for normal, sensitive, and highly sensitive responders; P<0.001). The mean±SD final warfarin dose was 5.1±2.1, 3.3±1.5, and 1.8±0.9 mg per day across genotype (P<0.001). During the first 90 days of warfarin treatment, sensitive and highly sensitive responders experienced higher rates of bleeding as compared with normal responders (HR 1.31, 95% CI 1.05-1.64, P=0.018 and HR 2.66, 95% CI 1.69-4.19, P<0.001). During this time period, when compared to warfarin, treatment with edoxaban resulted in a significantly decreased risk of bleeding in the sensitive and highly sensitive responders (P_{interaction}=0.004 and 0.007 for low and high edoxaban doses compared with warfarin). Thus, edoxaban provided better outcomes for treating and preventing bleeding events compared with warfarin, particularly in the medium and high sensitivity responders.

Thus, based on genotype, the sensitive and highly sensitive responders to warfarin required a lower dose of warfarin and less time to achieve a therapeutic INR, but spent a greater proportion of time over-anticoagulated with an INR >3, particularly through the first 90 days. In the 3-bin genotypic analysis of CYP2C9 and VKORC1 polymorphisms in the study, “sensitive” responders include “medium and moderately sensitive” responders, based on genotype. During this time period, sensitive responders experienced a 30% higher risk of bleeding as compared to normal responders, with highly sensitive responders demonstrating an over 2.5-fold increased risk of bleeding. Consequently, when compared to warfarin, edoxaban resulted in a particularly decreased risk of bleeding in sensitive and highly sensitive responders in this early period.

The current analysis from centers around the world includes close to 5000 subjects taking warfarin who were prospectively followed for an average of almost 3 years with central adjudication of bleeding events. The findings ultimately demonstrate the important contribution of the CYP2C9 and VKORC1 genes and validate the genetic binning used by the FDA. Moreover, the current analyses were performed in the context of a randomized, double-blinded trial, which tested two dosing regimens of edoxaban compared with warfarin in patients with atrial fibrillation, offering the opportunity to assess the impact of pharmacogenetics on the relative safety of the novel oral anticoagulant edoxaban compared with warfarin, based on genotype. Overall, in ENGAGE AF-TIMI 48, both doses of edoxaban, compared to warfarin, resulted in significantly lower rates of bleeding. The pharmacogenetic analysis demonstrates that the relative safety of edoxaban versus warfarin was particularly evident during the early time period among sensitive and highly sensitive responders to warfarin. After 90 days, the beneficial safety profile of edoxaban versus warfarin was evident across all the genetic categories.

As is often the case with clinical studies, some potential limitations of the study described herein are noted. First, this analysis included predominantly Caucasian patients, and as such, further analyses among other populations will be important. Second, this study was not designed to test the use of a pharmacogenetic versus a clinical algorithm for dose selection. However, the dose selection by the local investigator was based on the clinical profile of the subject with the use of an FDA recommended computer based algorithm, and the findings reflect current practices. Notably, the median achieved time-in-therapeutic range with warfarin in the ENGAGE-TIMI 48 study was 68.4%, which is high as compared to both the standard of care as well as clinical trial settings. Unlike the highly monitored conditions of a trial, in actual clinical practice, subjects taking warfarin may not be followed and dose-adjusted as closely or frequently as in a trial. Therefore, individuals who are genetically sensitive could experience a longer time in an over-anticoagulated state during the early phases of treatment with anticoagulant, when the appropriate dose ranges are still not yet wholly certain or finalized. Finally, for relatively rare events, such as life-threatening bleeding, the ability to definitively demonstrate or exclude a pharmacogenetic interaction was limited.

Several conclusions may be drawn from the ENGAGE-TIMI trial. Variants of the CYP2C9 and VKORC1 genes that are known to affect warfarin sensitivity had no effect on bleeding in subjects with AF treated with edoxaban. Using a 2-bin categorization, the relative rate of bleeding between the edoxaban and warfarin treatments was not significantly affected by genotype over the entire duration of the study. A statistically and clinically significant genotypic polymorphism effect was evident early in the course of anticoagulant therapy. During the first 90 days of treatment when the risk of bleeding is greatest, the relative rate of bleeding in edoxaban-treated subjects was significantly lower than that seen in warfarin-treated subjects having warfarin-sensitive genotypes. This effect was most pronounced in those subjects who were highly warfarin sensitive as determined using the three-bin system.

The variants in the VKORC1 and CYP2C9 genes have little to no effect on time to therapeutic range or time in therapeutic range over the entire duration of the study for warfarin-treated subjects; however, there is a substantial effect on time in therapeutic range during the first 90 days of treatment. These variants also have little or no effect on time on study drug over the entire duration of the study, or the likelihood of experiencing interruption or discontinuation of therapy, when viewed over the entire duration of the study during edoxaban or warfarin treatment. The VKORC1 genotype had no impact on the edoxaban pharmacodynamic response (i.e., PT, anti-FXa activity, and D-dimer biomarker levels for thromboembolism).

The results of this study provide strong evidence demonstrating that sensitive and highly sensitive responders based on CYP2C9 and VKORC1 genotypes have an increased exposure to warfarin, over-anticoagulation, and higher rates of bleeding. Among these patients, the beneficial safety profile of edoxaban compared with warfarin was particularly evident in the early time period, e.g., about 90 days into the treatment regimen.

All patents, patent applications and publications referred to or cited herein are hereby incorporated by reference in their entireties for all purposes.

It is understood that the embodiments and examples described herein are for illustrative purposes and that various modifications or changes in light thereof will be suggested to persons skilled in the pertinent art and are to be included within the spirit and purview of this application and scope of the appended claims. It is to be understood that suitable methods and materials are described herein for the practice of the embodiments; however, methods and materials that are similar or equivalent to those described herein can be used in the practice or testing of the invention and described embodiments.

TABLE 4
Baseline Characteristics Among WarfarinTreated Patients Across Genotype Bins

| | | | Highly | |
|---|---|---|---|---|
| | Normal | Sensitive | Sensitive | |
| | Responder | Responder | Responder | |
| Characteristic | (N = 2982) | (N = 1711) | (N = 140) | P Value |
| Median Age [interquartile range]-yr | 72 [64-78] | 72 [64-77] | 73 [66-78] | 0.46 |
| Male sex-no. (%) | 1818 (61.0) | 1071 (62.6) | 93 (66.4) | 0.28 |
| Race-no. (%) | | | | <0.001 |
| White | 2704 (90.7) | 1449 (84.7) | 129 (92.1) | |
| Asian | 139 (4.7) | 222 (13.0) | 9 (6.4) | |
| Black | 40 (1.3) | 2 (0.1) | 0 (0.0) | |
| All Others | 99 (3.3) | 38 (2.2) | 2 (1.4) | |
| Atrial fibrillation-no. (%) | | | | 0.68 |
| Paroxysmal | 798 (26.8) | 426 (24.9) | 35 (25.0) | |
| Persistent | 687 (23.0) | 395 (23.1) | 34 (24.3) | |
| Permanent | 1496 (50.2) | 890 (52.0) | 71 (50.7) | |
| Qualifying risk factors-no. (%) | | | | |
| Age ≧75 years | 1203 (40.3) | 657 (38.4) | 57 (40.7) | 0.41 |
| Prior stroke or transient ischemic attack | 850 (28.5) | 470 (27.5) | 36 (25.7) | 0.62 |
| Congestive heart failure | 1741 (58.4) | 969 (56.6) | 83 (59.3) | 0.47 |
| Diabetes mellitus | 1074 (36.0) | 647 (37.8) | 54 (38.6) | 0.42 |
| Hypertension requiring treatment | 2836 (95.1) | 1610 (94.1) | 132 (94.3) | 0.32 |
| CHADS2 score, Mean (±SD) | 2.9 ± 1.0 | 2.8 ± 1.0 | 2.8 ± 1.0 | 0.31 |
| ≦3 no. (%) | 2256 (75.7) | 1336 (78.1) | 110 (78.6) | 0.14 |
| 4-6-no. (%) | 726 (24.3) | 375 (21.9) | 30 (21.4) | |
| Creatinine clearance ≦50 ml/min-no. | 486 (16.3) | 330 (19.3) | 27 (19.3) | 0.03 |
| (%) | 195 (6.5) | 147 (8.6) | 9 (6.4) | 0.03 |
| Weight ≦60 kg-no. (%) | | | | |
| Previous vitamin K antagonist for ≧60 | | | | 0.48 |
| days-no. (%) | | | | |
| Current | 2085 (69.9) | 1210 (70.7) | 94 (67.1) | |
| Former | 156 (5.2) | 97 (5.7) | 5 (3.6) | |
| Never | 741 (24.8) | 404 (23.6) | 41 (29.3) | |
| Medications at time of randomization | | | | |
| no. (%) | | | | |
| Aspirin | 872 (29.3) | 453 (26.5) | 43 (30.7) | 0.10 |
| Thienopyridine | 66 (2.2) | 35 (2.0) | 3 (2.1) | 0.93 |
| Amiodarone | 341 (11.4) | 186 (10.9) | 19 (13.6) | 0.58 |
| Digoxin or digitalis preparations | 915 (30.7) | 518 (30.3) | 32 (22.9) | 0.14 |
| Region-no. (%) | | | | <0.001 |
| North America | 701 (23.5) | 373 (21.8) | 33 (23.6) | |
| Latin America | 242 (8.1) | 116 (6.8) | 5 (3.6) | |
| Western Europe | 458 (15.4) | 255 (14.9) | 27 (19.3) | |
| Eastern Europe | 1352 (45.3) | 708 (41.4) | 60 (42.9) | |
| Asia Pacific and South Africa | 229 (7.7) | 259 (15.1) | 15 (10.7) | |

TABLE 5
Adjusted Safety Outcomes During the First 90 Days Among
Warfarin Treated Patients Across Genotype Bins

| | | | Highly | |
|---|---|---|---|---|
| | Sensitive vs. | | Sensitive vs. | |
| | Normal | | Normal | |
| | Responders | P | Responders | P |
| Outcome | (HR) | Value | (HR) | Value |
| Any overt bleed | 1.27 (1.01-1.59) | 0.039 | 2.64 (1.67-4.17) | <0.001 |
| Major or | 1.22 (0.93-1.59) | 0.15 | 3.22 (1.99-5.21) | <0.001 |
| CRNM* | | | | |
| Major | 1.25 (0.73-2.15) | 0.42 | 2.14 (0.64-7.19) | 0.22 |
| CRNM | 1.20 (0.90-1.64) | 0.20 | 3.51 (2.12-5.58) | <0.001 |
| Intracranial | 1.20 (0.33-4.37) | 0.79 | 3.66 (0.43-30.99) | 0.23 |
| Life | 0.74 (0.19-2.94) | 0.67 | 3.46 (0.46-26.00) | 0.23 |
| Threatening | | | | |

*CRNM indicates “clinically relevant nonmajor” bleeding. Adjusted for covariates that differed across genotype bins (race, region, creatinine clearance, and weight) and previous exposure to a VKA.
HR indicates Hazard Ratio.

TABLE 6
Efficacy Outcomes Among Warfarin

| | 0-90 Days | | | | | | | | | |
|---|---|---|---|---|---|---|---|---|---|---|
| | Normal | | Medium | | | | High | | | |
| | Responder | | Sensitive | | Medium vs. | | Sensitive | | High vs. | |
| | (N = 2982) | | (N = 1711) | | Normal | | (N = 140) | | Normal | |
| | | KM | | KM | | 95% | | KM | | 95% |
| | n | Rates | n | Rates | HR | CI | n | Rates | HR | CI |
| Ischemic Stroke/ | 9 | 0.3 | 7 | 0.4 | 1.35 | (0.50- | 0 | 0.0 | — | — |
| SEE | | | | | | 3.63) | | | | |
| Isch. Stroke/SEE/ | 16 | 0.6 | 11 | 0.7 | 1.19 | (0.55 | 1 | 0.8 | 1.35 | (0.18- |
| Mortality | | | | | | 2.57) | | | | 10.17) |
| Ischemic Stroke | 7 | 0.2 | 7 | 0.4 | 1.74 | (0.61- | 0 | 0.0 | — | — |
| | | | | | | 4.95) | | | | |
| All-cause Death | 7 | 0.2 | 4 | 0.2 | 0.99 | 3.39) | 1 | 0.8 | 3.11 | (0.38- |
| | | | | | | (3.39) | | | | (25.19) |
| Death Due to | 7 | 0.2 | 4 | 0.2 | 0.99 | (0.29- | 1 | 0.8 | 3.11 | (0.38- |
| Cardiovascular | | | | | | 3.39) | | | | 25.19) |
| Causes | | | | | | | | | | |
| Myocardial | 3 | 0.1 | 5 | 0.3 | 2.89 | (0.69- | 0 | 0.0 | — | — |
| Infarction | | | | | | 12.10) | | | | |
| MACE | 18 | 0.6 | 16 | 1.0 | 1.54 | (0.79 | 1 | 0.8 | 1.20 | (0.16- |
| | | | | | | 3.03) | | | | 8.97) |

| | Beyond 90 Days | | | | | | | | | |
|---|---|---|---|---|---|---|---|---|---|---|
| | Normal | | Medium | | | | High | | | |
| | Responder | | Sensitive | | Medium vs. | | Sensitive | | High vs. | |
| | (N = 2803) | | (N = 1616) | | Normal | | (N = 129) | | Normal | |
| | | %/ | | %/ | | 95% | | %/ | | 95% |
| | n | yr | n | yr | HR | CI | n | yr | HR | CI |
| Ischemic Stroke/ | 58 | 0.9 | 34 | 0.9 | 1.02 | (0.67- | 3 | 1.0 | 1.11 | (0.35- |
| SEE | | | | | | 1.55) | | | | 3.52) |
| Isch. Stroke/SEE/ | 150 | 2.4 | 71 | 2.0 | 0.82 | (0.62- | 8 | 2.7 | 1.14 | (0.56- |
| Mortality | | | | | | 1.09) | | | | 2.32) |
| Ischemic Stroke | 53 | 0.8 | 30 | 0.8 | 0.98 | 0.63- | 3 | 1.0 | 1.22 | (0.38- |
| | | | | | | (1.54) | | | | 3.86) |
| All-cause Death | 100 | 1.6 | 37 | 1.0 | 0.64 | (0.44- | 5 | 1.7 | 1.07 | (0.43- |
| | | | | | | 0.93) | | | | 2.63) |
| Death Due to | 94 | 1.5 | 34 | 0.9 | 0.62 | (0.42- | 4 | 1.3 | 0.91 | (0.33- |
| Cardiovascular | | | | | | 0.92) | | | | 2.47) |
| Causes | | | | | | | | | | |
| Myocardial | 48 | 0.8 | 22 | 0.6 | 0.79 | (0.48 | 1 | 0.3 | 0.44 | (0.06- |
| Infarction | | | | | | 1.31) | | | | 3.17) |
| MACE | 185 | 2.95 | 87 | 2.4 | 0.82 | (0.63- | 8 | 2.7 | 0.92 | (0.46- |
| | | | | | | 1.05) | | | | 1.86) |

MACE indicated MI, ischemic stroke, SEE, and death due to cardiovascular causes; SEE, systemic embolic event.

TABLE 7
Intracranial Hemorrhage and Life Threatening Bleeding Among Warfarin and Edoxaban Treated
Patients Across Genotype

| | Event Rates | | | Edoxaban | |
|---|---|---|---|---|---|
| | (KM event rate) | | | High Dose vs. | Edoxaban Low |
| | Edoxaban | Edoxaban | | Warfarin | Dose vs. Warfarin |
| 0-90 Days | High Dose | Low Dose | Warfarin | HR (95% CI) | HR (95% CI) |
| Intracranial | | | | | |
| Bleed | | | | | |
| Normal | 0.1 | 0.1 | 0.2 | 0.70 (0.20-2.50) | 0.67 (0.19-2.39) |
| Responder | | | | | |
| Sensitive | 0.0 | 0.1 | 0.2 | — | 0.26 (0.03-2.30) |
| Highly | 0.0 | 0.0 | 0.8 | — | — |
| Sensitive | | | | | |
| Life- | | | | | |
| Threatening | | | | | |
| Bleed | | | | | |
| Normal | 0.2 | 0.2 | 0.2 | 0.90 (0.30-2.68) | 0.72 (0.23-2.28) |
| Responder | | | | | |
| Sensitive | 0.1 | 0.0 | 0.2 | 0.35 (0.04-3.32) | — |
| Highly | 0.0 | 0.0 | 0.8 | — | — |
| Sensitive | | | | | |
| | Event Rates | | | Edoxaban | |
| | (%/year) | | | High Dose vs. | Edoxaban Low |
| Beyond 90 | Edoxaban | Edoxaban | | Warfarin | Dose vs. Warfarin |
| Days | High Dose | Low Dose | Warfarin | HR (95% CI) | HR (95% CI) |
| Intracranial | | | | | |
| Bleed | | | | | |
| Normal | 0.4 | 0.2 | 0.6 | 0.75 (0.45-1.25) | 0.27 (0.14-0.55) |
| Responder | | | | | |
| Sensitive | 0.2 | 0.2 | 0.7 | 0.33 (0.15-0.72) | 0.27 (0.12-0.62) |
| Highly | 0.0 | 0.3 | 0.7 | — | 0.33 (0.03-3.48) |
| Sensitive | | | | | |
| Life- | | | | | |
| Threatening | | | | | |
| Bleed | | | | | |
| Normal | 0.4 | 0.2 | 0.4 | 1.04 (0.61-1.77) | 0.40 (0.20-0.81) |
| Responder | | | | | |
| Sensitive | 0.3 | 0.2 | 0.9 | 0.31 (0.15-0.63) | 0.24 (0.11-0.51) |
| Highly | 0.3 | 0.5 | 1.0 | 0.27 (0.03-2.51) | 0.48 (0.08-2.86) |
| Sensitive | | | | | |

1. A method of treating or preventing embolism, thrombosis, or thromboembolism in a subject in need thereof and identified as having one or more genetic polymorphisms in genes CYP2C9 and/or VKORC1 that result in warfarin sensitivity, the method comprising: administering to the subject a therapeutically effective amount of a pharmaceutical composition comprising a Factor Xa inhibitor.

2. A method of reducing the risk of bleeding event in a subject having a condition requiring oral anticoagulation therapy, the subject identified as having one or more genetic polymorphisms in genes CYP2C9 and/or VKORC1 that result in warfarin sensitivity, the method comprising: administering to the subject a therapeutically effective amount of a pharmaceutical composition comprising a Factor Xa inhibitor.

3. A method of treating or preventing drug-induced bleeding events or over-anticoagulation in a subject in need thereof, the subject identified as having one or more genetic polymorphisms in genes CYP2C9 and/or VKORC1 that result in warfarin sensitivity; the method comprising administering to the subject an effective amount of a Factor Xa inhibitor which is edoxaban or a pharmaceutically acceptable salt and/or hydrate thereof.

4. A method of guiding anticoagulant therapy by determining whether to administer warfarin or a Factor Xa inhibitor to a subject in need of anticoagulation therapy, the method comprising:
a) assaying a biological sample from the subject to identify genetic polymorphisms in genes CYP2C9 and/or VKORC1 resulting in warfarin sensitivity;
b) identifying the subject as carrying one or more genetic polymorphisms in the CYP2C9 and/or VKORC1 genes resulting in warfarin sensitivity;
c) if the subject is identified as carrying one or more of the genetic polymorphisms of step b), then administering a therapeutically effective amount of a composition comprising a Factor Xa inhibitor; or
d) if the subject is identified as not carrying any of the genetic polymorphisms of step b), then administering a therapeutically effective amount of a composition comprising warfarin, or a Factor Xa inhibitor, or warfarin or a VKA alternative drug or compound.

5. A method of treating thrombus, embolism, or thromboembolism in a subject to reduce the risk of a bleeding event, the method comprising:
a) assaying a biological sample from the subject to identify if the subject has sensitivity to warfarin;
b) identifying the subject as having sensitivity to warfarin; and
c) administering a therapeutic amount of a Factor Xa inhibitor to the subject identified as having sensitivity to warfarin in step b).

6. The method according to claim 1, wherein the subject is a human subject.

7. The method according to claim 1, wherein the subject is being treated for reducing the risk of stroke and/or systemic embolism in nonvalvular atrial fibrillation; for deep vein thrombosis (DVT); for pulmonary embolism (PE); for preventing or reducing the risk of recurrence of DVT and PE; for DVT following hip or knee replacement surgery; or for prophylaxis of DVT following hip or knee replacement surgery.

8. The method according to claim 1, wherein the one or more genetic polymorphisms in CYP2C9 and/or VKORC1 indicate that the subject is medium sensitive or highly sensitive to bleeding or over-anticoagulation associated with warfarin treatment.

9. The method according to claim 1, wherein the subject has one or more single nucleotide polymorphisms (SNPs) in alleles of the CYP2C9 gene and/or in alleles of the VKORC1 gene, the SNPs being associated with warfarin sensitivity in the subject.

10. The method according to claim 9, wherein the subject has one or more SNPs in alleles of the CYP2C9 gene selected from a single nucleotide polymorphism (SNP) in the *2 allele of CYP2C9 (rs1799853), a SNP in the *3 allele of CYP2C9 (rs1057910); and/or a −1639G>A (rs9923231) SNP genetic polymorphism in the VKORC1 gene, the SNPs being associated with warfarin sensitivity in the subject.

11. The method according to claim 1, wherein the subject has medium sensitivity to warfarin.

12. The method according to claim 11, wherein the subject has CYP2C9 and VKORC1 allelic genotypes selected from a *1/*1 genotype in CYP2C9 and an A/A genotype in VKORC1; a *1/*2 genotype in CYP2C9 and an A/G genotype in VKORC1; a *1/*2 genotype in CYP2C9 and an A/A genotype in VKORC1; a *1/*3 genotype in CYP2C9 and a G/G genotype in VKORC1; a *1/*3 genotype in CYP2C9 and an A/G genotype in VKORC1; a *2/*2 genotype in CYP2C9 and a G/G genotype in VKORC1; a *2/*2 genotype in CYP2C9 and an A/G genotype in VKORC1; or a *2/*3 genotype in CYP2C9 and a G/G genotype in VKORC1.

13. The method according to claim 1, wherein the subject has high sensitivity to warfarin.

14. The method according to claim 13, wherein the subject has CYP2C9 and VKORC1 allelic genotypes selected from a *1/*3 genotype in CYP2C9 and an A/A genotype in VKORC1; a *2/*2 genotype in CYP2C9 and an A/A genotype in VKORC1; a *2/*3 genotype in CYP2C9 and an A/G genotype in VKORC1; a *2/*3 genotype in CYP2C9 and an A/A genotype in VKORC1; a *3/*3 genotype in CYP2C9 and a G/G genotype in VKORC1; a *3/*3 genotype in CYP2C9 and an A/G genotype in VKORC1; or a *3/*3 genotype in CYP2C9 and an A/A genotype in VKORC1.

15. The method according claim 14, wherein the subject has an A/A genotype of VKORC1.

16. The method according to claim 1, wherein the Factor Xa inhibitor is a direct Factor Xa inhibitor.

17. The method according to claim 16, wherein the direct FXa inhibitor selected from edoxaban, rivaroxaban, LY517717, apixaban, 813893, betrixaban, AVE-3247, EMD-503982, WX-FX4, a pharmaceutically acceptable salt and/or hydrate thereof, or a combination thereof.

18. The method according to claim 1, wherein the Factor Xa inhibitor is edoxaban or a pharmaceutically acceptable salt and/or hydrate thereof.

19. The method according to claim 18, wherein the Factor Xa inhibitor is edoxaban tosylate monohydrate.

20. The method according to claim 16, wherein edoxaban is administered in an amount of 60 mg per day.

21. The method according to claim 16, wherein edoxaban is administered in an amount of 30 mg per day.

22. The method according to claim 1, wherein the administering comprises oral administration.

23. The method according to claim 1, wherein the Factor Xa inhibitor is in a solid oral form.

24. The method according to claim 1, wherein the Factor Xa inhibitor is an indirect FXa inhibitor.

25. The method according to claim 24, wherein the indirect FXa inhibitor is selected from heparins, heparinoids, low molecular weight (LMW) heparins, ultra-low molecular weight heparins, low molecular weight lignins (LMWLs), direct thrombin/Factor IIa inhibitors, or a combination thereof.

26. The method according to claim 1, wherein the Factor Xa inhibitor is administered in combination with another therapeutic agent, drug, or bioactive agent.

27. The method according to claim 26, wherein the therapeutic agent, drug, or bioactive agent is selected from statins, P-gp substrates, antiplatelet drugs; antithrombotic agents; fibrinolytics; non-steroidal anti-inflammatory drugs (NSAIDs); or proton pump inhibitors (PPIs).

28. The method according to claim 26, wherein the therapeutic or bioactive agent is not warfarin or a vitamin K antagonist drug or compound.

29. The method according to claim 5, wherein in step a) sensitivity to warfarin is identified by one or more genetic polymorphisms in CYP2C9 and/or VKORC1 genes.

30. The method according to claim 5, wherein in step a) sensitivity to warfarin is determined by screening for a warfarin sensitive phenotype in vivo or in vitro.

31. The method according to claim 29, wherein in step b), the subject is identified as carrying one or more genetic polymorphisms in the CYP2C9 and/or VKORC1 genes indicative of warfarin sensitivity.

32. The method according to claim 30, wherein in step b), the subject is identified as having loss of function, reduction in function, or aberrant function of one or both of the CYP2C9 and/or VKORC1 gene products.

33. The method according to claim 4, wherein the biological sample is a blood sample, a sputum sample, a buccal sample, a hair follicle sample, or a saliva sample.

34. The method according to claim 1, wherein the subject has, or is at risk of having, a condition or disorder selected from one or more of venous thromboembolism (VTE), deep vein thrombosis pulmonary embolism, embolism, thromboembolism (TE), and venous thrombosis (VT), cerebral infarction, cerebral embolism, myocardial infarction, angina pectoris, pulmonary infarction, pulmonary embolism (PE), Buerger's disease, deep venous thrombosis (DVT), disseminated intravascular coagulation syndrome, thrombus formation after surgery, thrombus formation after valve or joint replacement, thrombus formation and reocclusion after angioplasty, systemic inflammatory response syndrome (SIRS), multiple organ dysfunction syndrome (MODS), thrombus formation during extracorporeal circulation, blood clotting, or a recurrence or combination thereof.

35. The method according to claim 1, wherein warfarin or a vitamin K antagonist drug or compound is not administered to the subject.
